(19) United States
(12) Patent Application Publication
(10) Pub. No.: US 2024/0085436 A1
(43) Pub. Date: Mar. 14, 2024

(54) TEST KIT FOR TESTING BIOMARKERS OF ALZHEIMER DISEASE AND THE METHOD THEREOF

(71) Applicant: HANGZHOU CELLGENE BIOTECH CO.,LTD, Hangzhou (CN)

(72) Inventors: Yanqing ZHOU, Hangzhou (CN); Tao WANG, Hangzhou (CN); Chenhuai XU, Hangzhou (CN)

(21) Appl. No.: 18/466,212

(22) Filed: Sep. 13, 2023

(30) Foreign Application Priority Data
Sep. 14, 2022 (CN) 2022111160117
Aug. 30, 2023 (CN) 2023111107302

(51) Int. Cl. G01N 33/68; G01N 15/14; G01N 21/64

(52) U.S. Cl. CPC G01N 33/6896; G01N 15/14; G01N 21/6428; G01N 2015/149; G01N 2021/6419; G01N 2800/2821

(57) ABSTRACT

The present invention provides a kit of a related marker for a central neurodegenerative disease and a detection method thereof. The kit uses a double antibody sandwich detection method, and can simultaneously detect 5 Alzheimer disease-related proteins in the same sample. An A microsphere labeled with a rabbit antibody is adopted, shaking is conducted in an incubation process, and an incubating agent, including polyol, a polymer, a nonionic surfactant and a chelating agent, is added. The aforementioned components cooperate with each other to achieve a long-term effect of maintaining suspension of the microspheres, so as to promote the reaction in the incubation process, reduce the formation of microsphere agglomeration and reduce non-specific binding. The Fc fragment of a detection antibody is removed, and thus the interference with a non-specific reaction is excluded. The aforementioned improvements improve the accuracy in detection of the relevant proteins of Alzheimer disease, make the clinical detection application be more convenient, can reduce the cost of reagents and labor, and has strong specificity, high sensitivity and good repeatability, and thus has good application prospect.

### CROSS-REFERENCE TO RELATED APPLICATIONS

The present application claims the priority of a Chinese prior application No. 202211116011.7 filed on Sep. 14, 2022, and a Chinese prior application No. 202311110730.2 filed on Aug. 30, 2023, the specification, claims, abstract and drawings of which are all incorporated by reference as a part of itself.

### BACKGROUND OF THE INVENTION

### Field of the Invention

The present invention relates to the technical field of flow cytometry, and in particular to a kit of a related marker for a central neurodegenerative disease and a preparation method thereof.

### Description of the Related Art

Alzheimer disease (AD), commonly known as senile dementia, is a progressive central neurodegenerative disease with insidious onset. A typical histopathological change of the Alzheimer disease is senile plaques caused by deposition of an amyloid β-protein (Aβ), neurofibrillary tangles caused by abnormal phosphorylation of a Tau protein, and loss of neurons and synapses. According to a hypothesis of cascade of amyloid proteins, Aβ pathology is an upstream event of the AD, which drives the occurrence of tau pathology and neurodegeneration in neocortex. Aβ1-42, Aβ1-40, T-Tau and P-Tau are widely accepted AD biomarkers at home and abroad. Studies have shown that the expression of Aβ1-42 and Aβ1-42/Aβ1-40 in the plasma of an AD patient is down-regulated, while the expression of T-Tau and p-Tau-181 is up-regulated.

Aβ1-42 and Aβ1-40 are produced by hydrolysis of an amyloid precursor protein (APP) by BACE and γ-secretase, which can be gradually accumulated outside the membrane to form an Aβ protein polymer and to generate a toxic effect on neurons, leading to neuronal degeneration. Compared with Aβ1-40, Aβ1-42 has a very high agglutination, and it has been gradually accumulated in the early stage of senile plaque formation. The ratio of contents of Aβ1-42 to Aβ1-42/Aβ1-40 in the plasma reflects the pathological condition of Aβ in the brain, which can be used for early evaluation of the risk of suffering from AD dementia and mild cognitive impairment (MCI). Among them, Aβ1-42 will decrease even before Amyloid-D positron emission tomography (Aβ-PET).

A Tau protein is a microtubule-associated protein (MAP), the content of which is high in neurons of a central nervous system, and its main function is to regulate the stability of axonal microtubules. It is also a phosphoprotein with multiple phosphorylation sites, among which p-Tau-181, p-Tau-217 and the like are the most studied. Phosphorylated Tau will compete with tubulin to bind with a normal Tau protein and other MAPs, which will lead to microtubule depolymerization and form paired helical filaments (PHF) in the neurons, affecting the normal physiological function of the Tau protein. Studies have shown that the concentration of plasma T-Tau will also increase in a Creutzfeldt-Jakob disease (CJD) and a frontotemporal lobar dementia (FTD), which is an important predictor of CJD. A plasma p-Tau-181 level can reflect the pathological conditions of Aβ and Tau in AD, distinguish the Alzheimer disease from other neurodegenerative diseases (e.g., Parkinson's disease and vascular dementia, etc.), and monitor the disease progression of the AD patient throughout the clinical process.

An α-synuclein is a soluble protein with a size of 14 kDa, which is mainly expressed at a presynaptic terminal of the brain, and has functions of regulating the stability of a neuronal membrane, influencing presynaptic signal transduction and membrane trafficking, etc. Similar to the tau protein, the α-synuclein is also prone to pathological misfolding and aggregation, which leads to the occurrence of some neurodegenerative diseases, such as Parkinson's disease (PD), Lewy body dementia (DLB) and multiple system atrophy (MSA), etc. These diseases are also collectively referred to as an α-synuclein disease. The combined detection of the α-synuclein and the AD biomarker can further distinguish the AD from the α-synuclein disease.

In view of the above, it can be seen that the combined detection of Aβ1-42, Aβ1-40, T-Tau and p-Tau-181 can improve the accuracy of AD determination, and the α-synuclein disease can be ruled out by addition of the α-synuclein. Therefore, the combined detection of the concentrations of the Aβ1-42, Aβ1-40, T-Tau, p-Tau-181 and α-synuclein in human plasma can be used for auxiliary diagnosis of the AD.

Currently, the main methodology for quantitative detection of the Aβ1-42, Aβ1-40, T-Tau, p-Tau-181 and α-synuclein in the market is an enzyme-linked immunosorbent assay. The enzyme-linked immunosorbent assay has complicated manual operation steps and poor result repeatability. Immunity transmission turbidity is easy to operate and suitable for an ordinary automated biochemical analyzer and an ordinary spectrophotometer, but its shortcomings are poor sensitivity and precision, a large amount of required plasma and a long detection period. Currently, the detection technologies of the Aβ1-42, Aβ1-40, T-Tau, p-Tau-181 and α-synuclein are generally separate detection, which has poor performances such as reagent sensitivity and precision, a long detection time and complicated operations. Therefore, there is an urgent need for a reagent with excellent performance that can simultaneously detect several AD markers.

Therefore, it is necessary to provide a novel kit and a preparation method thereof to solve the aforementioned problems existed in the prior art.

### BRIEF SUMMARY OF THE INVENTION

Aiming at the problems existed in the prior art, the present invention provides a kit of a related marker for a central neurodegenerative disease and a preparation method thereof, wherein the kit is used for detecting a protein related to Alzheimer disease in human plasma. In the kit of the present invention, a double antibody sandwich detection method is used, in which a combination of highly-relevant proteins of Alzheimer disease is selected for detection, and the detection of at least 3 relevant proteins of Alzheimer disease can be completed in the same sample at the same time, which improves the accuracy in detection of the relevant proteins of Alzheimer disease, make the clinical detection application be more convenient, can reduce the cost of reagents and labor, and has strong specificity, high sensitivity and good repeatability, and thus has good application prospect.

In the present invention, the type of an antibody for labeling a microsphere is screened, and it has been found that only labeling with a murine antibody and labeling with a rabbit antibody can realize detection, and the effect of the labeling with a rabbit antibody is significantly better than that of the labeling with a murine antibody. The microsphere labeled with the rabbit antibody has stronger specificity and higher detection sensitivity. The detection limits of Aβ1-40, Aβ1-42, T-Tau, p-Tau-181 and α-synuclein are decreased after it is replaced with the rabbit antibody (the antibody conjugated to the microsphere is a rabbit monoclonal antibody). A possible reason for the aforementioned results is in that: the rabbit antibody can identify an epitope on a human antigen that is not immunogenic in rodents, which increases the total number of epitopes that can be targeted; the rabbit antibody has a strong immune response to a small molecule and a hapten, which is not common in rodents; inbred strains of rabbits are more rare, while most mouse strains are inbred, so that the rabbit has more diversity of immune response; and the rabbit uses an unique mechanism to genetically produce and diversify antibodies, making them have high affinity and specificity.

In the present invention, microspheres are screened, and it has been found that an A microsphere (a 5 μm carboxyl microsphere) has the best effect on detecting Aβ1-42, Aβ1-40, T-Tau, p-Tau-181, and α-synuclein in the present invention. The determination result at a low concentration is accurate with a lower quantitative limit, and the determination precision at a high concentration is high, so that the overall linear interval is larger.

In the present invention, an incubation process of microspheres is explored and studied, and it has been found that in the incubation process, shaking can keeping the microspheres be suspended to a certain extent without settling, and thus the reaction in the incubation process is promoted, thereby improving the detection effect and improving the detection sensitivity. However, the effect of the shaking is related to the physical and chemical properties of the microspheres and proteins. In the present invention, it can generate a beneficial effect of reducing the detection limits for T-Tau, P-Tau-181 and α-synuclein, but has no significant effect on detection of Aβ1-40 and Aβ1-42.

In the present invention, an incubating agent is added in the incubation process, and the incubating agent includes polyol, a polymer, a nonionic surfactant and a chelating agent. The addition of the polyol, polymer and nonionic surfactant can significantly reduce the detection limits for Aβ1-40 and Aβ1-42, and the addition of the chelating agent can further improve the accuracy and sensitivity of the overall detection result, and especially has a significant effect of reducing the detection limits for Aβ1-40 and Aβ1-42.

A polyol substance can increase the density of a preservation solution, so that the microspheres can exist in a suspension form, so as to reduce the probability of agglomeration and cross-linking of the microspheres, thereby in turn achieving the effect of promoting occurrence of the reaction, and avoiding the problem that because of agglomeration of immune microspheres or after a too long agglomeration time, the immune microspheres are firmly conjoined and are difficult to be dispersed again through shaking.

The nonionic surfactant, also called an amphoteric surfactant, can be adsorbed on a site that is not completely closed on the surface of a coated immune microsphere, thereby improving the steric hindrance on the surface of the immune microsphere, further reducing the non-specific adsorption of other proteins, and meanwhile improving the dispersibility of the microsphere. It cooperates with the polyol to reduce the formation of microsphere agglomeration, thereby improving the accuracy and sensitivity of the detection result.

The polymer can wrap or polymerize interfering proteins in serum and plasma samples together, thereby eliminating the interference with macromolecular proteins such as fibrin, making it impossible for impure proteins to interact with antigens/antibodies through weak interaction, and meanwhile reducing the possible non-specific antigen/antibody binding between samples and microspheres.

The chelating agent can promote the dispersion of microspheres and maintain a dispersed system structure, and cooperate with the aforementioned components to achieve a long-term effect of maintaining suspension of the microspheres, thereby improving the accuracy and sensitivity of the detection result, wherein sodium hyaluronate also has an additional effect: increasing the viscosity of an aqueous agent to increase the difficulty in deposition and attenuate the sedimentation rate, which makes sodium citrate in cooperation with sodium hyaluronate produce the best effect.

A Fc fragment of an antibody is a constant region of the antibody. In this constant region, the amino acid sequence presents the same state in different antibodies, and the different types of antibodies are further determined by the specific structure of the Fc fragment of the antibody. The Fc fragment of the antibody is a small fragment on an immunoglobulin. After the immunoglobulin binds with a corresponding antigen to form a complex, the complex needs to be processed in vivo. An indicator for the processed complex is the Fc fragment of the antibody, and in vivo phagocytes can recognize the Fc fragment of the antibody, which will play its own role after recognized. An antibody is a basically Y-shaped protein, which binds to an antigenic epitope on the Y, and a vertical column under the Y is called Fc, i.e., a crystallizable fragment. The antibody is a protein, which is a substance that can specifically bind with an antigen after the antigen stimulates the body, and the Fc fragment of the antibody is a part of the antibody. A structural change of the Fc fragment of the antibody is that after the immunoglobulin binds to the antigen, various biological effects will be generated. The complex of the antigen and the antibody has a certain mediating effect on cells through a Fc receptor, and plays a great role in an immune function.

In the present invention, through a large number of experimental studies, it has been found that in the detection process of the present invention, non-specific interference will have a great influence on the detection. In the research process of the present invention, a non-specific sample is diluted and then tested, and the ratio of the tested concentration of the sample to the tested concentration of the stock solution is obviously different from the dilution multiple. The non-specific sample is tested by using an isotype control antibody, and the tested concentration of the non-specific sample is obviously higher than the background concentration, which indicates that the non-specific reaction is mainly induced by the Fc fragment of the antibody.

In the present invention, through experimental researches, only by removing the Fc fragments of the Aβ1-40 antibody, Aβ1-42 antibody, p-Tau-181 antibody and T-Tau antibody, the interference with the aforementioned non-specific reactions can be eliminated to the greatest extent, so as to improve the sensitivity; however, for the α-synuclein antibody, the sensitivity is instead decreased possibly due to the change in the protein configuration of the antibody.

In an aspect, the present invention provides a kit for detecting an Alzheimer disease-related protein in human blood, which includes a solution of an antibody (first antibody to the protein)-conjugated microsphere, a biotin-conjugated antibody(second antibody to the protein) and an incubating agent. The incubating agent includes sorbitol, polyvinylpyrrolidone, Triton X-100, sodium citrate and sodium hyaluronate.

The antibody, including the first and second antibody, is one or more of an Aβ1-40 antibody, an Aβ1-42 antibody, a p-Tau-181 antibody, a T-Tau antibody and an α-synuclein antibody. Or the antibody including the first and second antibody consists of these five types of the antibody against to Aβ1-40, Aβ1-42, p-Tau-181, T-Tau and α-synuclein. The antibody of the first or second can specificity bind the five proteins of the sample, the five proteins are: Aβ1-40, Aβ1-42, p-Tau-181, T-Tau and α-synuclein. The first antibody and the second antibody can bind the different site of the target protein separately.

Further, the antibody-conjugated microsphere is an A microsphere which is a 5 μm carboxyl microsphere, including an A1 microsphere, an A2 microsphere, an A3 microsphere, an A4 microsphere and an A5 microsphere, and the fluorescence intensities of the A1-A5 microspheres are different.

Further, the Aβ1-40 antibody is conjugated with the A1 microsphere, the Aβ1-42 antibody is conjugated with the A2 microsphere, the p-Tau-181 antibody is conjugated with the A3 microsphere, the T-Tau antibody is conjugated with the A4 microsphere, and the α-synuclein antibody is conjugated with the A5 microsphere; and the A1-A5 microspheres are 5 μm carboxyl microspheres with different fluorescence intensities.

Further, the antibody conjugated to the microsphere is a rabbit monoclonal antibody.

Further, Fc fragments are removed from the Aβ1-40 antibody, the Aβ1-42 antibody, the p-Tau-181 antibody and the T-Tau antibody.

Further, a fluorescent reagent is further included. The fluorescent reagent is a biotin-BSA-SA-PE, which is a conjugate of streptavidin and phycoerythrin crosslinked with biotin-BSA.

Further, a blocking agent is further included, which is an inactivated murine IgG and an anti-HAMA polyclonal antibody.

Further included are a sample diluting solution, a reaction buffer and a washing buffer. The sample diluting solution includes a tris(hydroxymethyl)aminomethane buffer, sodium chloride and Proclin300. The reaction buffer solution includes a tris(hydroxymethyl)aminomethane buffer, sodium chloride, bovine serum albumin, a blocking agent, Proclin300 and Tween-20. The washing buffer includes a phosphate buffer and Tween-20.

Further, it further includes a calibration product and a quality control.

In another aspect, the present invention provides a method for detecting the aforementioned kit, which includes the following steps:
S1: sequentially adding a reaction buffer, a solution of an antibody-conjugated microsphere and a sample to be tested into a sample tube;
S2: adding a biotin-conjugated antibody and an incubating agent into the mixture obtained in the step S1, and incubating the obtained mixture at 2-8° C. with protection from light and shaping;
S3: adding a fluorescent reagent into that mixture obtained in the step S2, and incubating the obtained mixture at room temperature with protection from light and shaping to obtain a first complex; and
S4: detecting the fluorescence type and fluorescence signal intensity of the first complex on a flow cytometer, and calculating to obtain the contents of Aβ1-42, Aβ1-40, T-Tau, p-Tau-181 and α-synuclein in the sample to be tested.

In some embodiments, a frequency of the shaking is 300-500 r/min.

In a further aspect, the present invention provides use of an incubating agent in preparation of a formulation for keeping a microsphere be suspended, wherein the incubating agent includes sodium citrate and sodium hyaluronate.

The present invention has the following beneficial effects.

1. In the kit of the present invention, a double antibody sandwich detection method is used, in which a combination of highly-relevant proteins of Alzheimer disease is selected for detection, and the detection of at least 3 relevant proteins of Alzheimer disease can be completed in the same sample at the same time, which improves the accuracy in detection of the relevant proteins of Alzheimer disease, make the clinical detection application be more convenient, can reduce the cost of reagents and labor, and has strong specificity, high sensitivity and good repeatability, and thus has good application prospect.

2. In the present invention, the labelling antibody of the microsphere is screened, and it has been found that a microsphere labelled with a rabbit antibody has stronger specificity and higher sensitivity, and significantly reduces the detection limits of Aβ1-40, Aβ1-42, T-Tau, p-Tau-181 and α-synuclein.

3. In the present invention, microspheres are screened, and it has been found that the A microsphere has the best effect on detecting Aβ1-42, Aβ1-40, T-Tau, p-Tau-181, and α-synuclein in the present invention. The determination result at a low concentration is accurate with a lower quantitative limit, and the determination precision at a high concentration is high, so that the overall linear interval is larger.

4. In the present invention, an incubation process of microspheres is explored and studied, and it has been found that in the incubation process, shaking can keeping the microspheres be suspended to a certain extent without settling, so as to reduce the occurrence of microsphere agglomeration and promote the reaction in the incubation process, thereby improving the detection effect and improving the detection sensitivity, and it can generate a beneficial effect of reducing the detection limits for detection of T-Tau, P-Tau-181 and α-synuclein.

5. In the present invention, an incubating agent is added during the incubation process, including polyol, a polymer, a nonionic surfactant and a chelating agent. The aforementioned components cooperate with each other to achieve a long-term effect of maintaining suspension of the microspheres, so as to promote the reaction in the incubation process, reduce the formation of microsphere agglomeration, and reduce the possible non-specific antigen/antibody binding between the sample and the microsphere, thereby improving the accuracy and sensitivity of the detection result.

6. In the present invention, removing the Fc fragment of the detection antibody eliminates the interference with a non-specific reaction, thereby improving the accuracy and sensitivity of the detection result.

### BRIEF DESCRIPTION OF THE DRAWINGS

FIG. 1a is a schematic diagram of a detection principle of removing a Fc fragment from a detection antibody in a kit according to an example of the present invention;

FIG. 1b is a schematic diagram of a detection principle of not removing a Fc fragment from a detection antibody in a kit according to an example of the present invention;

FIG. 2 shows the distribution of antibody-conjugated microspheres according to an example of the present invention;

FIG. 3 is a calibration curve of flow fluorescence detection of Aβ1-40, Aβ1-42, T-Tau, p-Tau-181 and α-synuclein according to the third example of the present invention;

FIG. 4 shows a linear evaluation result of Aβ1-40 according to an example of the present invention;

FIG. 5 shows a linear evaluation result of Aβ1-42 according to an example of the present invention;

FIG. 6 shows a linear evaluation result of T-Tau according to an example of the present invention;

FIG. 7 shows a linear evaluation result of p-Tau-181 according to an example of the present invention;

FIG. 8 shows a linear evaluation result of α-synuclein according to an example of the present invention;

FIG. 9 is a comparison chart of the detected amount of Aβ1-40 between healthy people and AD patients according to an example of the present invention;

FIG. 10 is a comparison chart of the detected amount of Aβ1-42 between healthy people and AD patients according to an example of the present invention;

FIG. 11 is a comparison chart of the detected amount of T-Tau between healthy people and AD patients according to an example of the present invention;

FIG. 12 is a comparison chart of the detected amount of p-Tau-181 between healthy people and AD patients according to an example of the present invention;

FIG. 13 is a comparison chart of the detected amount of α-synuclein between healthy people and AD patients according to an example of the present invention; and

FIG. 14 is a comparison chart of the detected amount of a ratio of Aβ1-42/Aβ1-40 between healthy people and AD patients according to an example of the present invention.

### DETAILED DESCRIPTION OF THE INVENTION

In order to make the objectives, technical solutions and advantages of the present invention clearer, the following clearly and completely describes the technical solutions in the embodiments of the present invention with reference to the accompanying drawings of the present invention, the described embodiments are a part rather than all of the embodiments of the present invention. All other embodiments obtained by those of ordinary skills in the art based on the embodiments of the present invention without creative efforts are within the claimed scope of the present invention. The technical or scientific terms used herein shall have the usual meanings understood by those of ordinary skill in the art to which the present invention belongs, unless otherwise defined. The “comprise(s)/comprising” or “include(s)/including” and the like similar words as used herein mean that the elements or articles appearing before the word encompass the elements or articles listed after the word and their equivalents, and do not exclude other elements or articles.

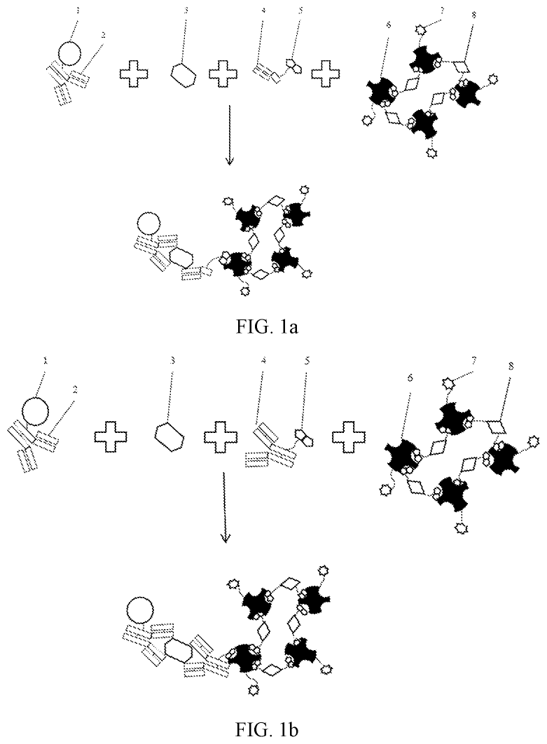

FIGS. 1a and 1b are schematic diagrams showing the relationship among a specific antibody, a microsphere, a detection antibody and phycoerythrin according to an example of the present invention.

Referring to FIG. 1a, a polystyrene microsphere 1 conjugated with an antibody protein 2, a sample to be tested 3, a detection antibody 4 conjugated with biotin 5, and a reticulate structure formed from a conjugate of streptavidin 6 and phycoerythrin 7 and a biotin-conjugated BSA 8 bind to form an immune complex, wherein the polystyrene microsphere 1 is labeled with a rabbit antibody, and the biotin 5 is conjugated with an antibody of which the Fc fragment has been removed. The immune complex is irradiated by two excitation lights with different wavelengths emitted by a flow cytometer. The types of detection indicators are determined by the fluorescence intensities of microspheres conjugated with different antibodies, and the contents of detection indicators are determined by the fluorescence intensity of phycoerythrin.

Referring to FIG. 1b, a polystyrene microsphere 1 conjugated with an antibody protein 2, a sample to be tested 3, a detection antibody 4 conjugated with biotin 5, and a reticulate structure formed from a conjugate of streptavidin 6 and phycoerythrin 7 and a biotin-conjugated BSA 8 bind to form an immune complex, wherein the polystyrene microsphere 1 is labeled with a rabbit antibody, and the biotin 5 is conjugated with an antibody of which the Fc fragment has not been removed. The immune complex is irradiated by two excitation lights with different wavelengths emitted by a flow cytometer. The types of detection indicators are determined by the fluorescence intensities of microspheres conjugated with different antibodies, and the contents of detection indicators are determined by the fluorescence intensity of phycoerythrin.

### Example 1: Preparation, Use and Effect Evaluation of Kit

I. Preparation of Kit

According to different detection indicators, the following could be prepared: 1) a first kit: a kit for simultaneously detecting Aβ1-42, Aβ1-40 and p-Tau-181, the specific composition of which was shown in Table 1; 2) a second kit: a kit for simultaneously detecting Aβ1-42, Aβ1-40, p-Tau-181 and α-synuclein, the specific composition of which was shown in Table 2; and 3) a third kit: a kit for simultaneously detecting Aβ1-42, Aβ1-40, T-Tau, p-Tau-181 and α-synuclein, the specific composition of which was shown in Table 3.

TABLE 1
Composition of the First Kit

| Reagent | | Loading |
|---|---|---|
| composition | Component | capacity |
| Solution of | a microsphere conjugated with an Aβ1-40 antibody, | 0.6 mL/bottle × 1 |
| antibody- | a microsphere conjugated with an Aβ1-42 antibody, | |
| conjugated | and a microsphere conjugated with a p-Tau-181 | |
| microsphere | antibody, | |
| Biotin- | a biotin conjugated with the Aβ1-40 antibody, a | 0.6 mL/bottle × 1 |
| conjugated | biotin conjugated with the Aβ1-42 antibody and a | |
| antibody | biotin conjugated with the p-Tau-181 antibody | |
| Fluorescent | A conjugate of streptavidin and phycoerythrin | 0.6 mL/bottle × 1 |
| reagent | crosslinked with biotin-BSA. | |
| Sample | A tris(hydroxymethyl)aminomethane buffer, sodium | 5 mL/bottle × 1 |
| diluting | chloride, and Proclin300 | |
| solution | | |
| Reaction | A tris(hydroxymethyl)aminomethane buffer, sodium | 0.6 mL/bottle × 1 |
| buffer | chloride, bovine serum albumin, a blocking agent | |
| | (inactivated murine IgG, an anti-HAMA polyclonal | |
| | antibody), Proclin300, and Tween-20 | |
| Washing | A phosphate buffer, Tween-20 | 3.5 mL/bottle × 1 |
| buffer (10×) | | |
| Incubating | Sorbitol, polyvinylpyrrolidone, Triton X-100, | 0.6 mL/bottle × 1 |
| agent | sodium citrate, sodium hyaluronate and a | |
| | tris(hydroxymethyl)aminomethane buffer | |
| Calibration | Freeze-dried products of Aβ1-40, Aβ1-42 | 1 mL after redissolution, the |
| product | and p-Tau-181 proteins | concentrations of respective |
| | | components were |
| | | Aβ1-40: 1250 pg/mL |
| | | Aβ1-42: 625 pg/mL |
| | | p-Tau-181: 160 pg/mL |
| Quality | Freeze-dried products of Aβ1-40, Aβ1-42 | 1 mL after redissolution, the |
| control I | and p-Tau-181 proteins | concentrations of respective |
| | | components were |
| | | Aβ1-40: 39.08 pg/mL |
| | | Aβ1-42: 19.52 pg/mL |
| | | p-Tau-181: 5 pg/mL |
| Quality | Freeze-dried products of Aβ1-40, Aβ1-42 | 5 mL after redissolution, the |
| control II | and p-Tau-181 proteins | concentrations of respective |
| | | components were |
| | | Aβ1-40: 312.5 pg/mL |
| | | Aβ1-42: 156.25 pg/mL |
| | | p-Tau-181: 40 pg/mL |

TABLE 2
Composition of the Second Kit

| Reagent | | Loading |
|---|---|---|
| composition | Component | capacity |
| Solution of | a microsphere conjugated with an Aβ1-40 antibody, a | 0.6 mL/bottle × 1 |
| antibody- | microsphere conjugated with an Aβ1-42 antibody, a | |
| conjugated | microsphere conjugated with a p-Tau-181 antibody, | |
| microsphere | and a microsphere conjugated with an α-synuclein | |
| | antibody | |
| Biotin- | a biotin conjugated with the Aβ1-40 antibody, a | 0.6 mL/bottle × 1 |
| conjugated | biotin conjugated with the Aβ1-42 antibody, a biotin | |
| antibody | conjugated with the p-Tau-181 antibody, and a biotin | |
| | conjugated with the α-synuclein antibody | |
| Fluorescent | A conjugate of streptavidin and phycoerythrin | 0.6 mL/bottle × 1 |
| reagent | crosslinked with biotin-BSA. | |
| Sample | A tris(hydroxymethyl)aminomethane buffer, sodium | 5 mL/bottle × 1 |
| diluting | chloride, and Proclin300 | |
| solution | | |
| Reaction | A tris(hydroxymethyl)aminomethane buffer, sodium | 0.6 mL/bottle × 1 |
| buffer | chloride, bovine serum albumin, a blocking agent, | |
| | Proclin300, and Tween-20 | |
| Washing | In terms of a phosphate buffer, Tween-20 | 3.5 mL/bottle × 1 |
| buffer (10×) | | |
| Incubating | Sorbitol, polyvinylpyrrolidone, Triton X-100, | 0.6 mL/bottle × 1 |
| agent | sodium citrate, sodium hyaluronate and a | |
| | tris(hydroxymethyl)aminomethane buffer | |
| Calibration | Freeze-dried products of Aβ1-40, Aβ1-42, | 1 mL after redissolution, the |
| product | p-Tau-181, and α-synuclein proteins | concentrations of respective |
| | | components were |
| | | Aβ1-40: 1250 pg/mL |
| | | Aβ1-42: 625 pg/mL |
| | | p-Tau-181: 160 pg/mL |
| | | α-synuclein: 32000 pg/mL |
| Quality | Freeze-dried products of Aβ1-40, Aβ1-42, | 1 mL after redissolution, the |
| control I | p-Tau-181, and α-synuclein proteins | concentrations of respective |
| | | components were |
| | | Aβ1-40: 39.08 pg/mL |
| | | Aβ1-42: 19.52 pg/mL |
| | | p-Tau-181: 5 pg/mL |
| | | α-synuclein: 1000 pg/mL |
| Quality | Freeze-dried products of Aβ1-40, Aβ1-42, | 5 mL after redissolution, the |
| control II | p-Tau-181, and α-synuclein proteins | concentrations of respective |
| | | components were |
| | | Aβ1-40: 312.5 pg/mL |
| | | Aβ1-42: 156.25 pg/mL |
| | | p-Tau-181: 40 pg/mL |
| | | α-synuclein: 8000 pg/mL |

TABLE 3
Composition of the Third Kit

| Reagent composition | Component | Loading capacity |
|---|---|---|
| Solution of antibody- | a microsphere conjugated with the | 0.6 mL/bottle × 1 |
| conjugated microsphere | Aβ1-40 antibody, a microsphere | |
| | conjugated with the Aβ1-42 | |
| | antibody, a microsphere | |
| | conjugated with the T-Tau | |
| | antibody, a microsphere | |
| | conjugated with the p-Tau-181 | |
| | antibody, and a microsphere | |
| | conjugated with the α-synuclein | |
| | antibody | |
| Biotin-conjugated | a biotin conjugated with the Aβ1- | 0.6 mL/bottle × 1 |
| antibody | 40 antibody, a biotin conjugated | |
| | with the Aβ1-42 antibody, a biotin | |
| | conjugated with the T-Tau | |
| | antibody, a biotin conjugated with | |
| | the p-Tau-181 antibody, and a | |
| | biotin conjugated with the α- | |
| | synuclein antibody | |
| Fluorescent reagent | A conjugate of streptavidin and | 0.6 mL/bottle × 1 |
| | phycoerythrin crosslinked with | |
| | biotin-BSA. | |
| Sample diluting | A | 5 mL/bottle × 1 |
| solution | tris(hydroxymethyl)aminomethane | |
| | buffer, sodium chloride, and | |
| | Proclin300 | |
| Reaction | A | 0.6 mL/bottle × 1 |
| buffer | tris(hydroxymethyl)aminomethane | |
| | buffer, sodium chloride, bovine | |
| | serum albumin, a blocking agent, | |
| | Proclin300, and Tween-20 | |
| Washing | In terms of a phosphate buffer, | 3.5 mL/bottle × 1 |
| buffer (10×) | Tween-20 | |
| Incubating | Sorbitol, polyvinylpyrrolidone, | 0.6 mL/bottle × 1 |
| agent | Triton X-100, sodium citrate, | |
| | sodium hyaluronate and a | |
| | tris(hydroxymethyl)aminomethane | |
| | buffer | |
| Calibration | Freeze-dried products of Aβ1-40, | 1 mL after redissolution, the |
| product | Aβ1-42, T-Tau, p-Tau-181, and α- | concentrations of respective |
| | synuclein proteins | components were |
| | | Aβ1-40: 1250 pg/mL |
| | | Aβ1-42: 625 pg/mL |
| | | p-Tau-181: 160 pg/mL |
| | | α-synuclein: 32000 pg/mL |
| | | T-Tau: 1250 pg/mL |
| Quality | Freeze-dried products of Aβ1-40, | 1 mL after redissolution, the |
| control I | Aβ1-42, T-Tau, p-Tau-181, and α- | concentrations of respective |
| | synuclein proteins | components were |
| | | Aβ1-40: 39.08 pg/mL |
| | | Aβ1-42: 19.52 pg/mL |
| | | p-Tau-181: 5 pg/mL |
| | | α-synuclein: 1000 pg/mL |
| | | T-Tau: 39.04 pg/mL |
| Quality | Freeze-dried products of Aβ1-40, | 5 mL after redissolution, the |
| control II | Aβ1-42, T-Tau, p-Tau-181, and α- | concentrations of respective |
| | synuclein proteins | components were |
| | | Aβ1-40: 312.5 pg/mL |
| | | Aβ1-42: 156.25 pg/mL |
| | | p-Tau-181: 40 pg/mL |
| | | α-synuclein: 8000 pg/mL |
| | | T-Tau: 312.5 pg/mL |

Taking the third kit for simultaneously detecting Aβ1-42, Aβ1-40, T-Tau, p-Tau-181 and α-synuclein as an example, its preparation method was as follows, and the first kit and the second kit could be obtained only by reducing respective reagents according to different detection indicators:

(1) Preparation of a Solution of an Antibody-Conjugated Microsphere

a first fluorescent microsphere, a second fluorescent microsphere, a third fluorescent microsphere, a fourth fluorescent microsphere and a fifth fluorescent microsphere were taken. Table 4 showed the manufacturers, models and fluorescence intensities of respective raw materials for preparing the antibody-conjugated fluorescent microspheres.

TABLE 4
Microsphere

| | | | Fluorescence |
|---|---|---|---|
| Microsphere | Factory | Model | intensity |
| The first fluorescent | Biolegend | 740167 | 4000 |
| microsphere (A1 microsphere) | | | |
| The second fluorescent | Biolegend | 740168 | 20000 |
| microsphere (A2 microsphere) | | | |
| The third fluorescent | Biolegend | 740169 | 50000 |
| microsphere (A3 microsphere) | | | |
| The fourth fluorescent | Biolegend | 740170 | 110000 |
| microsphere (A4 microsphere) | | | |
| The fifth fluorescent | Biolegend | 740171 | 140000 |
| microsphere (A5 microsphere) | | | |

The following anti-human Aβ1-42 antibody, anti-human Aβ1-40 antibody, anti-human T-Tau antibody, anti-human p-Tau-181 antibody and anti-human α-synuclein antibody were all rabbit monoclonal antibodies. The anti-human Aβ1-42 antibody was purchased from ABclonal. The anti-human Aβ1-40 antibody was purchased from Cell Signaling Technology. The anti-human T-Tau antibody and the anti-human α-synuclein antibody were purchased from Thermo. The anti-human p-Tau-181 antibody was purchased from abeam. The Aβ1-40 antibody was conjugated with an A1 microsphere, the Aβ1-42 antibody was conjugated with an A2 microsphere, the p-Tau-181 antibody was conjugated with an A3 microsphere, the T-Tau antibody was conjugated with an A4 microsphere, and the α-synuclein antibody was conjugated with an A5 microsphere. The A1-A5 microspheres were 5 μm carboxyl microspheres with different fluorescence intensities, and the fluorescence intensities were as shown in Table 4.

The steps for preparing the microsphere conjugated with the Aβ1-40 antibody were: 5×10⁶ first fluorescent microspheres were taken and washed with addition of a PBST buffer (a phosphate buffer, containing 0.05% TWEEN-20, pH 7.4, purchased from sigma) twice. The washed first fluorescent microspheres were added with 100 μg of EDC (N-(3-dimethylaminopropyl)-N′-ethylcarbodiimide hydrochloride, purchased from sigma) and 50 μg of NHS (a N-hydroxythiosuccinimide sodium salt, purchased from Aladdin) and allowed to stand for 30 minutes to activate the microspheres, added with 100 μg of an anti-human Aβ1-40 antibody, reacted with rotation at room temperature for 5 hours, and washed to remove excess antibody. After washed to remove excess anti-human Aβ1-40 antibody, the first fluorescent microsphere was then added with skimmed milk powder with a mass fraction of 5% for blocking for 30 minutes, and then added with a Tris buffer with a pH of 7.2 for preservation after the blocking solution was removed.

The steps for preparing the microsphere conjugated with the Aβ1-42 antibody were: 5×10⁶ second fluorescent microspheres were taken and washed with addition of the PBST buffer twice. The washed second fluorescent microspheres were added with 100 μg of EDC and 50 g of NHS and allowed to stand for 30 minutes to activate the microspheres, added with 100 μg of an anti-human Aβ1-42 antibody, reacted with rotation at room temperature for 5 hours, and washed to remove excess antibody. After washed to remove excess anti-human Aβ1-42 antibody, the second fluorescent microspheres were added with skimmed milk powder with a mass fraction of 5% for blocking for 30 minutes, and then added with a Tris buffer with a pH of 7.2 for preservation after the skimmed milk powder was removed.

The steps for preparing the microsphere conjugated with the p-Tau-181 antibody were: 5×10⁶ third fluorescent microspheres were taken and washed with addition of the PBST buffer twice. The washed third fluorescent microspheres were added with 100 μg of EDC and 50 μg of NHS and allowed to stand for 30 minutes to activate the microspheres, added with 100 μg of an anti-human p-Tau-181 antibody, reacted with rotation at room temperature for 5 hours, and washed to remove excess antibody. After washed to remove excess anti-human p-Tau-181 antibody, the third fluorescent microspheres were added with skimmed milk powder with a mass fraction of 5% for blocking for 30 minutes, and then added with a Tris buffer with a pH of 7.2 for preservation after the skimmed milk powder was removed.

The steps for preparing the microsphere conjugated with the α-synuclein antibody included: 5×10⁶ fourth fluorescent microspheres were taken and washed with addition of the PBST buffer twice. The washed fourth fluorescent microspheres were added with 100 μg of EDC and 50 μg of NHS and allowed to stand for 30 minutes to activate the microspheres, added with 100 μg of an anti-human α-synuclein antibody, reacted with rotation at room temperature for 5 hours, and washed to remove excess antibody. After washed to remove excess anti-human α-synuclein antibody, the fourth fluorescent microspheres were added with skimmed milk powder with a mass fraction of 5% for blocking for 30 minutes, and then added with a Tris buffer with a pH of 7.2 for preservation after the skimmed milk powder was removed, so as to prepare the microsphere conjugated with the α-synuclein antibody.

The steps for preparing the microsphere conjugated with the T-Tau antibody were: 5×10⁶ fifth fluorescent microspheres were taken and washed with addition of the PBST buffer twice. The washed fifth fluorescent microspheres were added with 100 μg of EDC and 50 μg of NHS and allowed to stand for 30 minutes to activate the microspheres, added with 100 μg of an anti-human T-Tau antibody, reacted with rotation at room temperature for 5 hours, and washed to remove excess antibody. After washed to remove excess anti-human T-Tau antibody, the fifth fluorescent microspheres were added with skimmed milk powder with a mass fraction of 5% for blocking for 30 minutes, and then added with a Tris buffer with a pH of 7.2 for preservation after the skimmed milk powder was removed.

98 mL of a Tris buffer with a pH of 7.2 was taken, and added with each 0.5 mL of the microsphere conjugated with the Aβ1-40 antibody, the microsphere conjugated with the Aβ1-42 antibody, the microsphere conjugated with the T-Tau antibody, the microsphere conjugated with the p-Tau-181 antibody and the microsphere conjugated with the α-synuclein antibody respectively according to the detection indicators of the kit, so as to formulate the solution of the antibody-conjugated microsphere.

(2) Preparation of Biotin-Conjugated Antibody

Fc fragments were removed from the purchased anti-human Aβ1-40 antibody, anti-human Aβ1-42 antibody, anti-human T-Tau antibody and anti-human p-Tau-181 antibody by a method as follows.
1. A 0.1 mol/L sodium acetate buffer with a pH=5, containing 0.1 mol/L of NaCl and 5 mg/mL of a dialyzed antibody, was used.
2. 0.3 mg of pepsin was dissolved into a dialyzed antibody solution, and cultured at 37° C. for 18 hours.
3. The pH was adjusted to 7.0, and gel filtration was conducted by using Sephacryl S-200HR with a 0.1 mol/L sodium phosphate buffer (pH=7.0) as an elution solution according to a speed of 0.35 mL/min.
4. The component F(ab′)₂ was taken out for concentration. The absorbance at 280 nm was measured to calculate the concentration. The product was added with 1% (V/V) sodium azide with a concentration of 100 mg/mL and stored for later use.
5. The aforementioned F(ab′)₂ was diluted to 0.4 mg/mL with a sodium phosphate buffer with a pH=6, and 0.9 mL of the diluted solution was taken and added with 0.1 mL of a 2-mercaptoethylamine solution. The mixture was cultured at 37° C. for 90 min.
6. Gel filtration was carried out with a sodium phosphate solution with a pH=6 (containing 5 mmol/l of EDTA) in an Ultrogel AcA44 column according to a speed of 0.35 mL/min.
7. The Fab′ component was taken out, and measured for the absorbance at 280 nm to calculate the concentration.

Each 100 μg of the treated anti-human Aβ1-40 antibody, anti-human Aβ1-42 antibody, anti-human T-Tau antibody, anti-human p-Tau-181 antibody and anti-human α-synuclein antibody, was taken, respectively added with a biotin according to a molar ratio of the antibody to biotin of 1:30, and incubated at room temperature for 5 hours to obtain the biotin-conjugated anti-human Aβ1-40 antibody, the biotin-conjugated anti-human Aβ1-42 antibody, the biotin-conjugated anti-human T-Tau antibody, the biotin-conjugated anti-human p-Tau-181 antibody and the biotin-conjugated anti-human α-synuclein antibody. Unbound biotins were removed, and then the biotin-conjugated anti-human Aβ1-40 antibody, the biotin-conjugated anti-human Aβ1-42 antibody, the biotin-conjugated anti-human T-Tau antibody, the biotin-conjugated anti-human p-Tau-181 antibody and the biotin-conjugated anti-human α-synuclein antibody were respectively diluted to 4 μg/ml with a 0.01 mol/L PBS solution.

Each 25 mL of the aforementioned biotin-conjugated anti-human Aβ1-40 antibody, biotin-conjugated anti-human Aβ1-42 antibody, biotin-conjugated anti-human T-Tau antibody, biotin-conjugated anti-human p-Tau-181 antibody and biotin-conjugated anti-human α-synuclein antibody with a concentration of 4 μg/mL was respectively taken and mixed to formulate the biotin-conjugated antibody. The concentration of each biotin-labeled antibody was 0.8 μg/mL.

(3) Preparation of a calibration product. Different concentrations of proteins Aβ1-40, Aβ1-42, T-Tau, p-Tau-181, and α-synuclein were diluted to different concentrations with a sample diluting solution, and then freeze-dried respectively and combined to obtain calibration products, which were used for establishing calibration curves during detection. See Table 3 for specific configurations. Table 5 showed the manufacturers and models of the proteins Aβ1-40, Aβ1-42, T-Tau, p-Tau-181 and α-synuclein as the calibration products.

TABLE 5
Protein sources

| Proteins as calibration products | Factory | Model |
|---|---|---|
| Aβ1-40 | Abcam | ab120479 |
| Aβ1-42 | Abcam | ab120301 |
| T-Tau | MilliporeSigma | T0576 |
| p-Tau-181 | Abcam | ab269022 |
| α-synuclein | MilliporeSigma | S7820 |

(4) Preparation of quality controls. The quality controls consisted of high-value and median-value freeze-dried recombinant proteins of Aβ1-40, Aβ1-42, T-Tau, p-Tau-181 and α-synuclein, so as to judge the reliability of the test results. See Table 3 for the specific configuration. Refer to Table 5 for the source of the quality control proteins.

(5) Preparation of the fluorescent reagent. SA-PE was purchased from thermofisher, diluted to 4 μg/mL for later use, and biotin-BSA was prepared by taking 100 μg of BSA, adding biotin into it according to a molar ratio of the BSA to biotin of 1:30, incubating at room temperature for 5 hours, then removing unbound biotin to obtain the biotin-conjugated BSA, and then diluting it into a solution containing 4 μg/mL of SA-PE according to 1 μg/mL of the biotin-conjugated BSA for later use.

(6) Preparation of the incubating agent. 6.057 g of tris(hydroxymethyl)aminomethane (Tris) was dissolved in 1,000 mL of pure water, then added with 9 g of sorbitol, 10 mL of polyvinylpyrrolidone, 5 g of Triton X-100, 12 g of sodium citrate and 20 g of sodium hyaluronate, and mixed for later use.

(7) Preparation of the sample diluting solution, the reaction buffer and the washing buffer:
the sample diluting solution was prepared by dissolving 9 g of sodium chloride (NaCl) and 6.057 g of tris(hydroxymethyl)aminomethane (Tris) in 1,000 mL of pure water, adding Proclin300 as a preservative with a mass concentration of 1%, and adjusting the pH to 7.4 for later use;
the reaction buffer was prepared by dissolving 9 g of NaCl and 6.057 g of Tris in 1,000 mL of pure water, adding bovine serum albumin (BSA) with a mass concentration of 1%, a blocking agent (inactivated murine IgG, anti-HAMA polyclonal antibody) with a mass concentration of 5%, Proclin300 as a preservative with a mass concentration of 0.1% and Tween-20 with a mass concentration of 0.1%, and adjusting the pH to 7.4 for later use; and
the washing buffer (10×) was prepared by dissolving 2.4 g of potassium dihydrogen phosphate (KH₂PO₄), 36.32 g of dipotassium hydrogen phosphate dodecahydrate (K₂HPO₄·12H₂O), 8 g of NaCl and 2 g of KCl in 1,000 mL of pure water, adding 25 g of BSA, Proclin300 as a preservative with a mass concentration of 1% and Tween-20 with a mass concentration of 0.5%, and mixing for later use.

II. Method for Using the Kit

After redundant detection indicators were removed, the methods for using the first and second kits were the same as that of the third kit. Taking the third kit as an example, the particular method for using the third kit was as follows:
into a sample tube, a calibration curve tube and a quality control tube added were 25 μl of a reaction buffer and 25 μL of a solution of an antibody-conjugated microsphere, mixed fully and evenly, and shaken with a shaker for more than 30 seconds;
into the sample tube added was a sample, into the calibration curve tube added was a calibration product, and into the quality control tube added was a quality control;
into the sample tube, the calibration curve tube and the quality control tube added were 25 μL of the biotin-conjugated antibody and 25 μL of the incubating agent;
the sample tube, the calibration curve tube and the quality control tube were refrigerated at 2-8° C. and incubated with protection from light under shaking at a shaking frequency of 300-500 r/min for 18 hours, and the shaking frequencies involved in the examples of the present invention are all 500 r/min;
into the sample tube, the calibration curve tube and the quality control tube added were the fluorescent, and incubated with protection from light under shaking at room temperature for 0.5 hour;
into the sample tube, the calibration curve tube and the quality control tube added were 1,000 μL of a 1× washing buffer, the microspheres were resuspended by vortex, mixed fully and evenly, shaken with a shaker for more than 30 seconds, and then centrifuged at 300 g for 5 minutes, and the supernatant was discarded; and
into the sample tube, the calibration curve tube and the quality control tube added were 150 μL-300 μL of the 1× washing buffer, the microspheres were resuspended by vortex, mixed fully and evenly, shaken with a shaker for more than 30 seconds, and detected for the fluorescence type and fluorescence signal intensity on a BriCyte E6 flow cytometer produced by Mindray.

The required sample size of the kit of the present invention was small, and only 25 μL of plasma was needed.

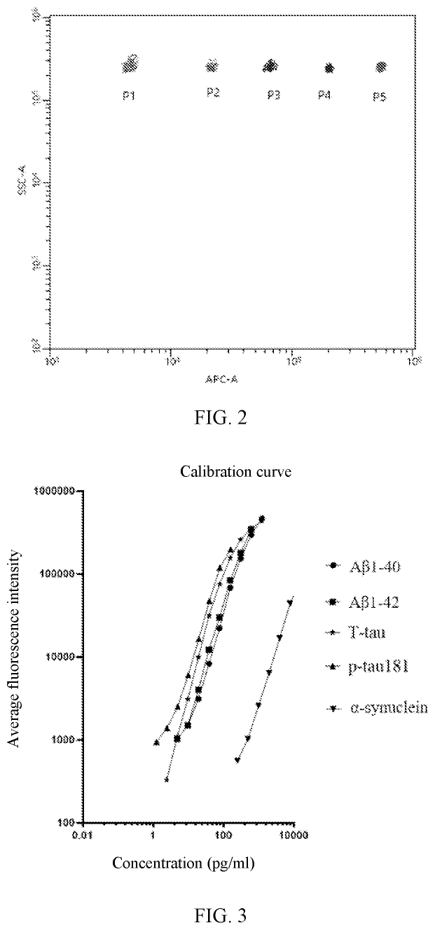

FIG. 2 showed the distribution of antibody-conjugated microspheres according to an example of the present invention. Referring to FIG. 2, side scatter light (SSC) was taken as the vertical coordinate and allophycocyanin (APC) was taken as the horizontal coordinate. The vertical coordinate determined a position interval of the microsphere complexity, and the horizontal coordinate distinguished the fluorescence intensities of APCs carried by different microspheres. FIG. 2 could distinguish four kinds of microspheres conjugated with the Aβ1-40, Aβ1-42, T-Tau, p-Tau-181 and α-synuclein antibodies, wherein P1 represented the SSC and APC distribution of the microspheres conjugated with the Aβ1-40 antibody in the example of the present invention; P2 represented the distribution of SSC and APC of the microspheres conjugated with the Aβ1-42 antibody in the example of the present invention, P3 represented the distribution of SSC and APC of the microspheres conjugated with the T-Tau antibody in the example of the present invention, P4 represented the distribution of SSC and APC of the microspheres conjugated with the p-Tau-181 antibody in the example of the present invention, and P5 represented the distribution of SSC and APC of the microspheres conjugated with the α-synuclein antibody in the example of the present invention.

III. Evaluation of Test Results of the Kit

The performances of the third kit in detecting Aβ1-40, Aβ1-42, T-Tau, p-Tau-181 and α-synuclein in a sample were evaluated. A specific performance evaluation method was as follows:

(a) Formulation of a Lx Washing Buffer:
(1) a 10× washing buffer was stabilized to room temperature; and
(2) the 10× washing buffer was diluted with purified water at a ratio of 1:9 (1 part of the 10× washing buffer added with 9 parts of purified water) into a 1× washing buffer for later use.

(b) Preparation of a Calibration Product
(1) a freeze-dried powder of a calibration product was added with 1 mL of a sample diluting solution to completely dissolve the freeze-dried powder;
(2) the solution was allowed to stand at room temperature for 15 minutes, and the concentration of this solution was the highest concentration, which was marked as C1;
(3) 11 empty tubes were prepared, and were marked as C10, C9, C8, C7, C6, C5, C4, C3, C2, C1 and C0 respectively;
(4) each tube was added with 75 μL of the sample diluting solution and diluted according to a diluting multiple of 2, that was, 75 μl of the C1 solution was taken and added into C2 and mixed thoroughly; and
(5) diluting was conducted in the same way to obtain C10, C9, C8, C7, C5, C4, C3, C2 and C1 calibration products respectively. C0 was added with only the sample diluting solution.

(c) Performance Evaluation

Into a sample tube, a calibration curve tube and a quality control tube added were 25 μl of a reaction buffer and 25 μL of a solution of an antibody-conjugated microsphere, mixed fully and evenly, and shaken with a shaker for more than 30 seconds;
into the sample tube added was a sample, into the calibration curve tube added was a calibration product, and into the quality control tube added was a quality control;
into the sample tube, the calibration curve tube and the quality control tube added were 25 μL of the biotin-conjugated antibody and 25 μL of the incubating agent;
the sample tube, the calibration curve tube and the quality control tube were refrigerated at 2-8° C. and incubated with protection from light for 18 hours;
into the sample tube, the calibration curve tube and the quality control tube added were the fluorescent, and incubated with protection from light at room temperature for 0.5 hour;
into the sample tube, the calibration curve tube and the quality control tube added were 1,000 μL of a 1× washing buffer, the microspheres were resuspended by vortex, mixed fully and evenly, shaken with a shaker for more than 30 seconds, and then centrifuged at 300 g for 5 minutes, and the supernatant was discarded; and
into the sample tube, the calibration curve tube and the quality control tube added were 150 μL-300 μL of the 1× washing buffer,
the microspheres were resuspended by vortex, mixed fully and evenly, shaken with a shaker for more than 30 seconds, and detected for the fluorescence type and fluorescence signal intensity on a BriCyte E6 flow cytometer produced by Mindray.

FIG. 3 was a calibration curve of flow fluorescence detection of Aβ1-40, Aβ1-42, T-Tau, p-Tau-181 and α-synuclein according to the third example of the present invention. Calibration products with different concentrations of Aβ1-40, Aβ1-42, T-Tau, p-Tau-181 and α-synuclein were respectively detected on the flow cytometer, and the calibration curve in FIG. 3 was obtained.

(d) Evaluation of Linearity, Blank Limit and Detection Limit

A high-concentration sample close to the upper limit of a linear range was diluted into samples with at least 5 different concentrations (xi), with each concentration being tested for 3 times, and the average values (yi) of the detection results were respectively calculated. With the diluted concentrations (xi) as independent variables and the average values (yi) of the test results as dependent variables, a correlation coefficient (R) of linear regression was calculated.

Method for detecting a blank limit: a calibration product with a concentration of zero was used as a sample for detection, and the determination was repeated for 20 times. The concentration values of the 20 measurements were obtained according to the curve equation of the calibration product used in the kit, and an average value (m) and a standard deviation (SD) thereof were calculated to get M+2SD, which was the blank limit.

Method for detecting a detection limit: 5 low-value samples with concentrations approximating the detection limit (the approximate detection limit was estimated according to the obtained blank limit, and was slightly higher than the blank limit) were detected, with each sample being detected for 5 times. The detection results were sorted according to the size, and the number of test results below the numerical value of the blank limit (the blank limits of Aβ1-42, Aβ1-40, T-Tau, p-Tau-181 and α-synuclein were 0.54 pg/mL, 0.57 pg/mL, 0.73 pg/mL, 0.37 pg/mL and 16.30 pg/mL, respectively) should be less than or equal to 3.

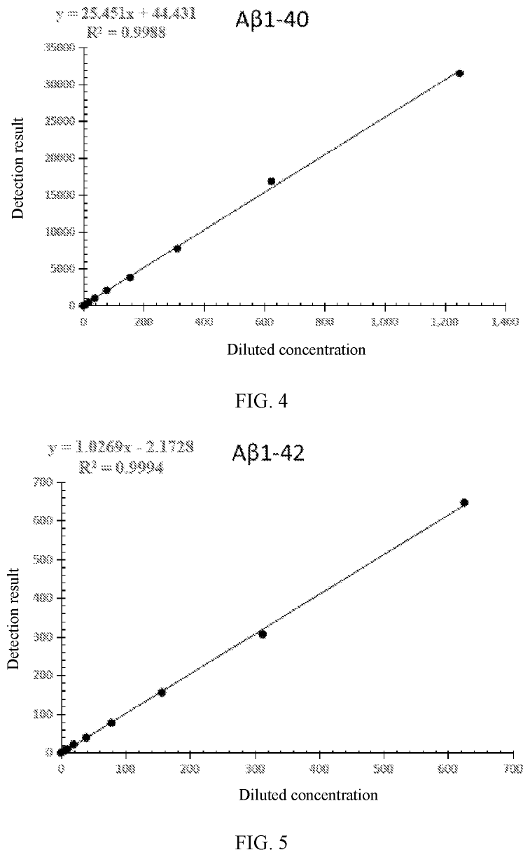

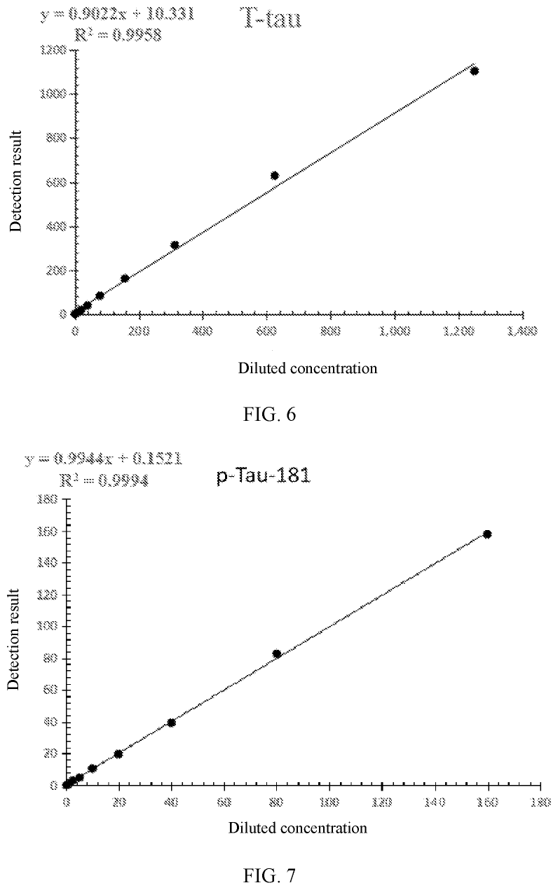

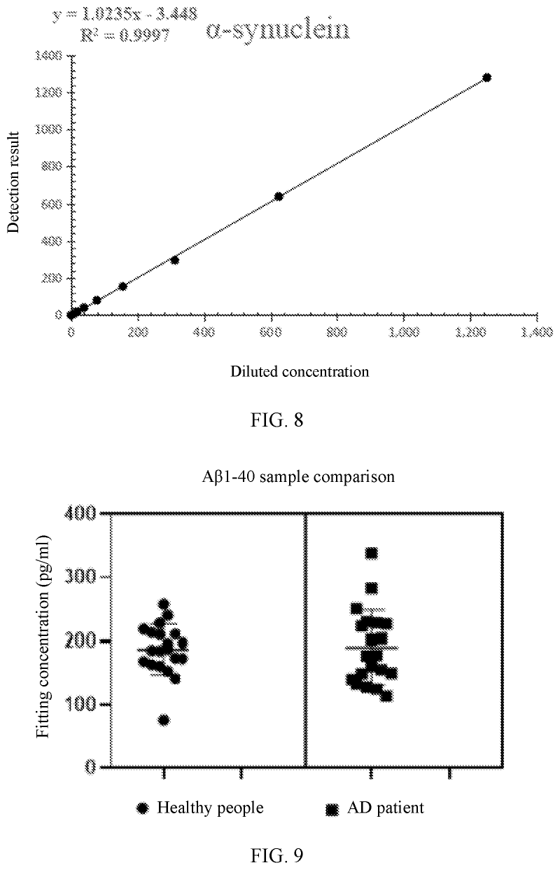

FIG. 4 showed a linear evaluation result of Aβ1-40 according to an example of the present invention; FIG. 5 showed a linear evaluation result of Aβ1-42 according to an example of the present invention; FIG. 6 showed a linear evaluation result of T-Tau according to an example of the present invention; FIG. 7 showed a linear evaluation result of p-Tau-181 according to an example of the present invention; and FIG. 8 showed a linear evaluation result of α-synuclein according to an example of the present invention. Referring to FIGS. 4-8, the linear regression equation of Aβ1-40 was y=25.451x+44.431, and R²=0.9988; the linear regression equation of Aβ1-42 was y=1.0269x−2.1728, and R²=0.9994; the linear regression equation of T-tau was y=0.9022x+10.331, and R²=0.9958; the linear regression equation of p-Tau-181 was y=0.9944x+0.1521, and R²=0.9994; and the linear regression equation of α-synuclein was y=1.0235x−3.448, and R²=0.9997.

With the kit of the present invention, it could be detected that a linear interval of Aβ1-40 was not narrower than [9.77, 1250] pg/mL, a linear interval of Aβ1-42 was not narrower than [4.88, 625] pg/mL, a linear interval of T-Tau was not narrower than [9.77, 1250] pg/mL, a linear interval of p-Tau-181 was not narrower than [1.25, 160] pg/mL, a linear interval of α-synuclein was not narrower than [250, 32000] pg/ml, and the linear correlation coefficient |R| was no less than 0.990.

Table 6 showed the results of a detection limit test (pg/mL). Referring to Table 10, the detection limits of Aβ1-40, Aβ1-42, T-Tau, p-Tau-181 and α-synuclein were 0.60 pg/mL, 0.64 pg/mL, 0.75 pg/mL, 0.44 pg/mL and 18.10 pg/mL, respectively. Therefore, the detection limits of Aβ1-40, Aβ1-42, T-Tau, p-Tau-181 and α-synuclein were not higher than 2.5 pg/mL, 2.5 pg/mL, 1.5 pg/ml, 1 pg/mL and 100 pg/mL respectively, and the detection sensitivity of the kit of the present invention was high.

TABLE 6
Detection Limit Test (pg/mL)

| Serial number | Aβ1-40 | Aβ1-42 | T-Tau | p-Tau-181 | α-synuclein |
|---|---|---|---|---|---|
| 1 | 0 | 0 | 0 | 0 | 0 |
| 2 | 0 | 0.44 | 0 | 0.42 | 0 |
| 3 | 0 | 0 | 0.44 | 0 | 9.77 |
| 4 | 0 | 0 | 0 | 0 | 0 |
| 5 | 0 | 0 | 0.48 | 0.18 | 0 |
| 6 | 0 | 0.68 | 0 | 0 | 10.58 |
| 7 | 0 | 0 | 0.92 | 0 | 0 |
| 8 | 0 | 0 | 0 | 0.55 | 0 |
| 9 | 0 | 0 | 0.69 | 0 | 0 |
| 10 | 0 | 0 | 0 | 0 | 0 |
| 11 | 1.09 | 0 | 0.08 | 0 | 12.28 |
| 12 | 0 | 0.77 | 0 | 0 | 0 |
| 13 | 0 | 0 | 0 | 0 | 0 |
| 14 | 0 | 0 | 0.3 | 0 | 0 |
| 15 | 0 | 0 | 0.4 | 0 | 0 |
| 16 | 0 | 0 | 0 | 0.12 | 24.69 |
| 17 | 0 | 0 | 0 | 0 | 0 |
| 18 | 0 | 0 | 0 | 0 | 0 |
| 19 | 0 | 0 | 0.33 | 0 | 7.2 |
| 20 | 0 | 0 | 0 | 0 | 0 |
| Average value | 0.05 | 0.09 | 0.18 | 0.06 | 3.23 |
| SD | 0.24 | 0.24 | 0.28 | 0.15 | 6.54 |
| Blank limit | 0.54 | 0.57 | 0.73 | 0.37 | 16.30 |
| Detection limit | 0.60 | 0.64 | 0.75 | 0.44 | 18.10 |

(e) Evaluation of Repeatability and Batch-to-Batch Difference

Repeatability: samples of Aβ1-40, Aβ1-42, T-Tau, p-Tau-181 and α-synuclein at high and low concentration levels were detected respectively, with each sample being repeatedly detected for 10 times. The average value M and standard deviation SD of the results of the 10 times of detection were calculated, and a coefficient of variation CV was calculated.

Table 7 showed the results of a repeatability test (pg/mL). Referring to Table 11, the coefficients of variation CV of the high-value and low-value calibration products of Aβ1-40, Aβ1-42, T-Tau, p-Tau-181 and α-synuclein were all within 10%.

TABLE 7
Repeatability Test (pg/mL)

| | Aβ1-40 | | Aβ1-42 | | T-Tau | | p-Tau-181 | | α-synuclein | |
|---|---|---|---|---|---|---|---|---|---|---|
| | Low | High | Low | High | Low | High | Low | High | Low | High |
| | concen- | concen- | concen- | concen- | concen- | concen- | concen- | concen- | concen- | concen- |
| | tration | tration | tration | tration | tration | tration | tration | tration | tration | tration |
| 1 | 38.53 | 343.62 | 21.35 | 140.62 | 8.30 | 297.60 | 4.63 | 40.86 | 941.37 | 84439.64 |
| 2 | 39.44 | 352.20 | 22.37 | 145.49 | 9.25 | 295.54 | 4.91 | 41.98 | 1002.95 | 86997.63 |
| 3 | 39.56 | 353.36 | 17.83 | 167.41 | 10.38 | 339.32 | 5.21 | 37.78 | 1021.56 | 78586.73 |
| 4 | 42.10 | 346.38 | 18.41 | 170.78 | 10.46 | 301.25 | 4.70 | 38.82 | 1098.33 | 77827.61 |
| 5 | 35.72 | 352.65 | 19.25 | 144.72 | 10.67 | 319.00 | 5.01 | 38.22 | 1096.79 | 72873.42 |
| 6 | 37.68 | 348.44 | 17.98 | 136.14 | 9.31 | 282.67 | 4.65 | 42.68 | 925.13 | 86787.02 |
| 7 | 38.99 | 336.60 | 20.82 | 158.94 | 8.94 | 333.86 | 4.62 | 37.74 | 957.93 | 81062.88 |
| 8 | 40.21 | 343.57 | 17.93 | 172.22 | 10.47 | 341.13 | 5.22 | 37.66 | 1096.70 | 79506.03 |
| 9 | 38.88 | 335.41 | 19.66 | 169.02 | 9.97 | 336.76 | 4.61 | 43.78 | 1052.20 | 74133.65 |
| 10 | 39.92 | 325.48 | 20.43 | 150.97 | 10.94 | 334.68 | 5.48 | 43.86 | 907.44 | 76818.62 |
| Average | 39.10 | 343.77 | 19.60 | 155.63 | 9.87 | 318.18 | 4.90 | 40.34 | 1010.04 | 79903.32 |
| value | | | | | | | | | | |
| SD | 1.67 | 8.98 | 1.60 | 13.67 | 0.87 | 21.91 | 0.31 | 2.58 | 74.50 | 4922.30 |
| CV | 4% | 3% | 8% | 9% | 9% | 7% | 6% | 6% | 7% | 6% |

Batch-to-batch difference: three batches of kits were taken, and were respectively used for detecting the same reference sample, with each detection being repeated for 10 times. An average value M and standard deviation SD of 30 measurements were calculated, and the coefficient of variation CV was calculated.

Table 8 showed the evaluation results of batch-to-batch difference (pg/mL). Referring to Table 12, the CVs of batch-to-batch differences of the 3 batches for Aβ1-40, Aβ1-42, T-Tau, p-Tau-181 and α-synuclein, were all within 10%.

TABLE 8
batch-to-batch difference (pg/mL)

| | Serial number | Aβ1-40 | Aβ1-42 | T-Tau | p-Tau-181 | α-synuclein |
|---|---|---|---|---|---|---|
| Batch I | 1 | 302.48 | 164.73 | 343.04 | 42.42 | 72015.15 |
| | 2 | 314.58 | 148.58 | 308.68 | 41.40 | 84707.97 |
| | 3 | 332.80 | 154.83 | 289.85 | 41.32 | 77389.11 |
| | 4 | 316.20 | 141.92 | 328.99 | 38.39 | 75102.62 |
| | 5 | 285.78 | 152.09 | 327.23 | 38.02 | 75549.51 |
| | 6 | 269.07 | 170.21 | 320.51 | 37.04 | 86430.64 |
| | 7 | 330.47 | 141.91 | 338.96 | 42.45 | 78974.58 |
| | 8 | 305.45 | 157.60 | 319.30 | 37.64 | 83178.25 |
| | 9 | 286.31 | 163.07 | 304.46 | 41.15 | 85475.64 |
| | 10 | 305.32 | 146.72 | 323.12 | 37.42 | 73612.80 |
| Batch II | 11 | 259.22 | 164.06 | 314.37 | 42.42 | 86746.92 |
| | 12 | 294.91 | 140.05 | 314.09 | 38.66 | 75926.72 |
| | 13 | 276.81 | 171.08 | 330.09 | 40.83 | 87674.04 |
| | 14 | 258.12 | 146.32 | 337.80 | 42.10 | 80354.89 |
| | 15 | 274.81 | 152.13 | 295.94 | 36.26 | 75206.16 |
| | 16 | 319.59 | 165.77 | 316.52 | 39.51 | 81445.13 |
| | 17 | 314.00 | 160.93 | 286.25 | 42.25 | 77588.75 |
| | 18 | 302.31 | 179.27 | 326.96 | 36.56 | 77651.16 |
| | 19 | 299.13 | 137.32 | 307.94 | 36.41 | 81504.82 |
| | 20 | 262.92 | 147.04 | 343.46 | 37.65 | 78386.86 |
| Batch III | 21 | 300.14 | 146.45 | 304.76 | 44.00 | 72146.33 |
| | 22 | 293.18 | 178.37 | 340.00 | 38.98 | 81595.59 |
| | 23 | 307.46 | 177.70 | 287.35 | 43.04 | 81617.26 |
| | 24 | 328.07 | 160.83 | 315.10 | 37.43 | 78631.65 |
| | 25 | 270.41 | 164.41 | 298.42 | 37.82 | 85641.02 |
| | 26 | 314.81 | 155.07 | 307.10 | 36.28 | 74059.91 |
| | 27 | 335.52 | 175.17 | 307.31 | 40.08 | 80209.36 |
| | 28 | 331.90 | 143.82 | 309.13 | 36.04 | 82987.53 |
| | 29 | 270.81 | 144.29 | 337.10 | 40.50 | 84503.44 |
| | 30 | 339.45 | 158.59 | 320.51 | 41.52 | 75060.29 |
| | Average value | 300.07 | 157.01 | 316.81 | 39.52 | 79712.47 |
| | SD | 24.31 | 12.34 | 16.50 | 2.42 | 4583.34 |
| | CV | 8% | 8% | 5% | 6% | 6% |

Through the evaluation of the repeatability and the batch-to-batch difference, it could be seen that the kit of the present invention had good repeatability and batch-to-batch difference, the coefficient of variation (CV) of intra-batch experiments was no more than 15%, and the CV of the batch-to-batch differences was no more than 15%.

It could be seen from the aforementioned examples that the kit for simultaneously detecting Aβ1-40, Aβ1-42, T-Tau, p-Tau-181 and α-synuclein by flow fluorescence technique as provided by the present invention could be used for detecting the concentrations of Aβ1-40, Aβ1-42, T-Tau, p-Tau-181 and α-synuclein in different samples, with a wide linear range, high precision, good specificity and stable determination results.

IV. Practical Application

Comparison and evaluation of the first, second and third kits for detecting an AD patient sample and a healthy sample. Each 20 samples of healthy people and AD patients aged 60-80 were respectively selected for comparative analysis.

The First Kit:

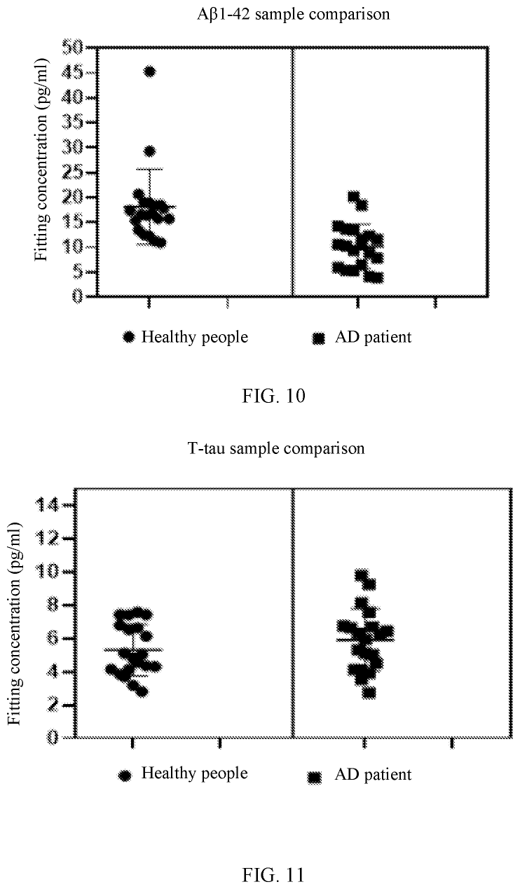

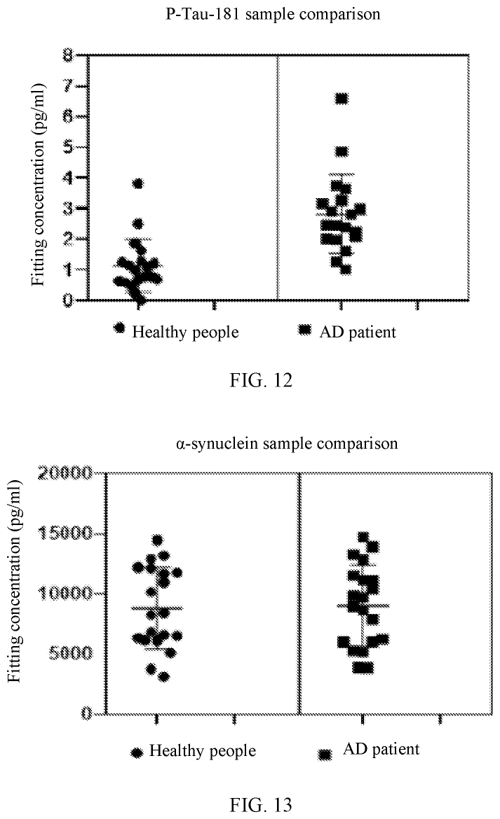

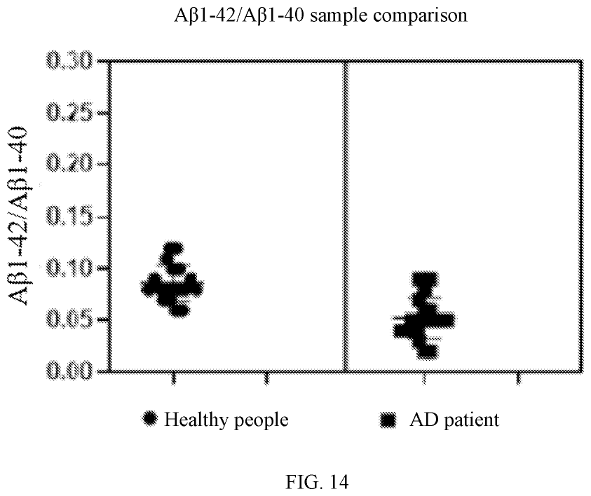

FIG. 9 was a comparison chart of the detected amount of Aβ1-40 between healthy people and AD patients according to an example of the present invention; FIG. 10 was a comparison chart of the detected amount of Aβ1-42 between healthy people and AD patients according to an example of the present invention; FIG. 12 was a comparison chart of the detected amount of p-Tau-181 between healthy people and AD patients according to an example of the present invention; and FIG. 14 was a comparison chart of the detected amount of a ratio of Aβ1-42/Aβ1-40 between healthy people and AD patients according to an example of the present invention. Referring to FIGS. 9, 10, 12 and 14, the medians of Aβ1-42 and the ratio of Aβ1-42/Aβ1-40 in the blood of the AD patients were 11.7 (5.8, 22.4) and 0.06 (0.02, 0.11), respectively, the medians of Aβ1-42 and the ratio of Aβ1-42/Aβ1-40 in the blood of the healthy people were 17.5 (11.4, 45.6) and 0.08 (0.06, 0.13), respectively, and thus the medians of Aβ1-42 and the ratio of Aβ1-42/Aβ1-40 in the blood of the AD patients had a reducing trend compared with those of the healthy people. The median of p-Tau-181 in the blood of the AD patients was 2.6 (0.8, 6.6), which had an increasing trend compared with the median 1.2 (0, 3.9) of p-Tau-181 in the blood of the healthy people. The kit of the present invention could simultaneously and more accurately detect Aβ1-40, Aβ1-42 and p-Tau-181 in the blood, which was more convenient for clinical application.

The Second Kit:

FIG. 13 was a comparison chart of the detected amount of α-synuclein between healthy people and AD patients according to an example of the present invention. Referring to FIGS. 9, 10, 12, 13 and 14, the medians of Aβ1-42 and the ratio of Aβ1-42/Aβ1-40 in the blood of the AD patients were 11.7 (5.8, 22.4) and 0.06 (0.02, 0.11), respectively, the medians of Aβ1-42 and the ratio of Aβ1-42/Aβ1-40 in the blood of the healthy people were 17.5 (11.4, 45.6) and 0.08 (0.06, 0.13) respectively, and thus the Aβ1-42 and the ratio of Aβ1-42/Aβ1-40 in the blood of the AD patients had a reducing trend compared with those of the healthy people. The median 2.6 (0.8, 6.6) of p-Tau-181 in the blood of the AD patients had an increasing trend compared with the median 1.2 (0, 3.9) of p-Tau-181 in the blood of the healthy people. The median of α-synuclein in the blood of the healthy people was 9885.4 (4,565.6, 14,862.3), which was basically equally to that of α-synuclein in the blood of the AD patients. The kit of the present invention could simultaneously and more accurately detect Aβ1-40, Aβ1-42, p-Tau-181 and α-synuclein, which was more convenient for clinical application.

The Third Kit:

FIG. 12 was a comparison chart of the detected amount of p-Tau-181 between healthy people and AD patients according to an example of the present invention. Referring to FIGS. 9-14, for Aβ1-42 and the ratio of Aβ1-42/Aβ1-40, the medians of Aβ1-42 and the ratio of Aβ1-42/Aβ1-40 in the blood of the AD patients were 11.7 (5.8, 22.4) and 0.06 (0.02, 0.11), respectively, the medians of Aβ1-42 and the ratio of Aβ1-42/Aβ1-40 in the blood of the healthy people were 17.5 (11.4, 45.6) and 0.08 (0.06, 0.13), respectively, and thus the Aβ1-42 and the ratio of Aβ1-42/Aβ1-40 in the blood of the AD patients had a reducing trend compared with those of the healthy people; for p-Tau-181, the median of p-Tau-181 in the blood of the AD patients was 2.6 (0.8, 6.6), which had an increasing trend compared with the median 1.2 (0, 3.9) of p-Tau-181 in the blood of the healthy people; and for T-tau and α-synuclein, the medians of T-tau and α-synuclein in the blood of the AD patients were 5.6 (2.5, 9.8) and 9763.1 (4565.6, 14667.7), respectively, the medians of T-tau and α-synuclein in the blood of the healthy people were 5.3 (2.6, 7.9) and 9885.4 (4565.6, 14862.3), respectively, and thus the T-tau and α-synuclein in the blood of the AD patients were basically equal to those of the healthy people. The kit of the present invention could simultaneously and more accurately detect Aβ1-40, Aβ1-42, T-tau, p-Tau-181 and α-synuclein, which was more convenient for clinical application.

### Example 2: Microspheres and Test for Screening Microspheres Coated with Antibody

The detection sensitivity of p-Tau-181 was not high in both the existing kit and the existing detection method, and the detection result was unstable. We tried to improve the detection of p-Tau-181 by replacing different microspheres, and the average fluorescence intensity was tested according to the method of Example 1 (except that the anti-human p-Tau-181 antibody was conjugated with different microspheres:A3 microspheres, Y7 microspheres and L10 microspheres). As shown in Table 9, the A3 microspheres were 5 μm carboxyl microspheres, the Y7 microspheres were 4 μm carboxyl microspheres and the L10 microspheres were 5 μm amino microspheres.

TABLE 9
Average fluorescence intensity of p-Tau-181 under
different microspheres (tested for 10 times)
p-Tau-181 MFI

| Concentration of | Y7 | L10 | A3 | | |
|---|---|---|---|---|---|
| P-Tau-181, pg/mL | microspheres | microspheres | microspheres | A3/Y7 | A3/L10 |
| 0 | 73 | 708 | 85 | 1.17 | 0.12 |
| 0.63 | 152 | 1300 | 533 | 3.5 | 0.41 |
| 1.25 | 562 | 1721 | 1067 | 1.9 | 0.62 |
| 2.5 | 1512 | 4774 | 2721 | 1.8 | 0.57 |
| 5 | 4400 | 8970 | 9239 | 2.1 | 1.03 |
| 10 | 10025 | 14655 | 26966 | 2.69 | 1.84 |
| 20 | 26945 | 28879 | 60357 | 2.24 | 2.09 |
| 40 | 64346 | 77301 | 115179 | 1.79 | 1.49 |
| 80 | 94784 | 93971 | 207273 | 2.19 | 2.21 |
| 160 | 148420 | 131967 | 325817 | 2.20 | 2.47 |

The results showed that the average fluorescence intensity of p-Tau-181 under the Y7 microspheres was not high, and thus was difficult to detect at a low concentration, and the sensitivity was low; the p-Tau-181 at low concentration under the L10 microspheres had large interference, and the detection result was inaccurate; and the A3 microspheres could relatively obviously increase the detection titer by about 1 time with high sensitivity.

Similarly, we tested the other antibody-conjugated microspheres, and had found that the 5 m carboxyl microspheres (the A microspheres) could improve the detection sensitivity, and the detection results showed the same trend as those in Table 9. In order to distinguish five detection indicators, we conjugated them to A microspheres with different fluorescence intensities respectively: A1 microspheres conjugated with the Aβ1-40 antibody, A2 microspheres conjugated with the Aβ1-42 antibody, A3 microspheres conjugated with the p-Tau-181 antibody, A4 microspheres conjugated with the T-Tau antibody, and A5 microspheres conjugated with the α-synuclein antibody.

At the same time, the present invention also selected a murine monoclonal antibody for a comparative test. The detection limits of Aβ1-40, Aβ1-42, T-Tau, p-Tau-181 and α-synuclein under microspheres conjugated with a murine antibody and microspheres conjugated with a rabbit antibody were determined according to the method of Example 2, and the results were as shown in Table 10.

TABLE 10
Screening of Antibodies for Coating Microspheres (pg/mL)

| | Aβ1-40 | Aβ1-42 | T-Tau | p-Tau-181 | α-synuclein |
|---|---|---|---|---|---|
| Microspheres | 1.28 | 1.33 | 1.78 | 1.05 | 33.74 |
| coated with a | | | | | |
| murine antibody | | | | | |
| Microspheres | 0.60 | 0.64 | 0.75 | 0.44 | 18.10 |
| coated with a | | | | | |
| rabbit antibody | | | | | |

The results showed that the microspheres labeled with the rabbit antibody had stronger specificity and higher detection sensitivity. After the microspheres were replaced with microspheres conjugated with the rabbit antibody, the detection limits of Aβ1-40, Aβ1-42, T-Tau, p-Tau-181 and α-synuclein were decreased, and the detection sensitivity was increased obviously.

The possible reasons for the aforementioned results were:
i. the rabbit antibody could recognize an epitope on a human antigen that was not immunogenic in a rodent, thereby increasing the total number of epitopes that could be targeted;
ii. the rabbit antibody had a strong immune response to small molecules and haptens, which was not common in the rodent;
iii. inbred strains of rabbits were rarer, while most mouse strains were inbred, so that the rabbits had more diversity of immune responses; and
iv. the rabbits used a unique mechanism to genetically produce and diversify antibodies, so that the antibodies had high affinity and specificity.

### Example 3: Test for Screening Incubation Conditions

As in Example 1, we needed to conduct incubation after the reaction buffer, the antibody-conjugated microspheres and the biotin-conjugated antibodies were added. The incubation conditions directly affected the adequacy of the reaction and the subsequent detection. If the reaction was insufficient, the subsequent average fluorescence intensity detected by the flow cytometer was inaccurate, resulting in inaccurate detection results as well. However, in the prior art, the incubation was often incubating at room temperature for a certain period of time after mixing, and the microspheres are easy to deposit, which in turn led to insufficient reaction.

The first improvement of the present invention lied in adjusting the incubation time and temperature; and we set up three groups of incubation schemes:
scheme 1: it was added with the reaction buffer, the microspheres, the calibration product protein and the detection antibody and then incubated at room temperature for 2 h, and then added with biotin-BSA-SA-PE and incubated for 30 min;
scheme 2: it was added with the reaction buffer, the microspheres, the calibration product protein and the detection antibody and then incubated at room temperature for 4 h, and then added with-BSA-SA-PE and incubated for 30 min; and
scheme 3: it was added with the reaction buffer, the microspheres, the calibration product protein and the detection antibody and then incubated at 2-8° C. overnight for 18 h, and then added with biotin-BSA-SA-PE and incubated for 30 min.

Other steps were the same as those in Example 1. The average fluorescence intensity was detected by the flow cytometer. The results of Aβ1-40, Aβ1-42, p-Tau-181 and T-Tau showed that the titer was obviously doubled after incubation at 2-8° C. for 18 h; the test result of α-synuclein after incubation at 2-8° C. for 18 h was consistent with that of incubation for 4 h; and thus, in order to improve the detection effect, the scheme 3 should be selected for incubation.

In the present invention, a great deal of research had been carried out on the method of adding a reagent during incubation, and it had been found that by labeling BSA with a biotin, and then binding the obtained biotin-BSA with SA-PE to form a reticular structure, a better effect could be obtained by incubating with a reagent of the aforementioned reticular structure. The trends of the five detection indicators were similar, and the average fluorescence intensity of p-Tau-181 could reflect the overall results. The detection results were as shown in Table 11.

TABLE 11
Average fluorescence intensity of P-tau-181 (tested for 10 times)
p-Tau-181 MFI

| Concentration | 4 μg/mL | 0.5 μg/mL of biotin- | 1 μg/mL of biotin- | 1.5 μg/mL of biotin- |
|---|---|---|---|---|
| of P-Tau-181, | of | BSA + 4 μg/mL | BSA + 4 μg/mL | BSA + 4 μg/mL |
| pg/mL | SA-PE | of SA-PE | of SA-PE | of SA-PE |
| 0 | 105.00 | 152.00 | 85 | 95.00 |
| 0.63 | 98.00 | 238.00 | 533 | 550.00 |
| 1.25 | 197.00 | 463.00 | 1067 | 958.00 |
| 2.50 | 525.00 | 1232.00 | 2721 | 2846.00 |
| 5.00 | 1714.00 | 4915.00 | 9239 | 10739.00 |
| 10.00 | 5065.00 | 11212.00 | 26966 | 25564.00 |
| 20.00 | 10950.00 | 33471.00 | 60357 | 51211.00 |
| 40.00 | 28998.00 | 54849.00 | 115179 | 132461.00 |
| 80.00 | 56687.00 | 109082.00 | 207273 | 217618.00 |
| 160.00 | 100075.00 | 148091.00 | 325817 | 309510.00 |

Conclusion: in the past, the biotin+SA-PE system is obtained by only binding a biotin on an antibody with SA-PE, and the amplification effect thereof was only that multiple biotins were bound on one antibody and then bound with the SA-PE, so that the redundant biotins and antibodies in the system failed to play their own role of stepwise amplification. In this project, during use the SA-PE bound with the biotin-labeled BSA in a proper proportion to form a reticular structure in advance, and then the reticular structure was added into the reaction system to amplify the value of the detection signal. BSA was labeled with biotin and then bound with SA-PE to form a reticular structure before use. According to the results of combination and formulation, the test result cooperated with the optimal concentration (1 μg/mL of biotin-BSA+4 g/mL of SA-PE) was about 4 times that of SA-PE alone.

On the basis of the aforementioned incubation method, we had found that shaking could keep the microspheres be suspended to a certain extent without settling, so that the reaction was more complete and the detection sensitivity could be improved. We compared the detection limits with shaking and without shaking according to the method of Example 2, with the shaking frequency being 500 r/min to ensure that the microspheres remained suspended to a certain extent without settling, and no incubating agent was added both with and without shaking. The results were as shown in Table 12.

TABLE 12
Optimization of Sensitivity of Incubation with shaking (pg/mL)

| | Aβ1-40 | Aβ1-42 | T-Tau | p-Tau-181 | α-synuclein |
|---|---|---|---|---|---|
| Incubation | 3.54 | 3.91 | 1.96 | 1.61 | 31.8 |
| without shaking | | | | | |
| Incubation | 3.51 | 3.87 | 1.40 | 0.98 | 23.3 |
| with shaking | | | | | |

Shaking did not necessarily improve the sensitivity in immunoquantitation, and its effect was related to the physical and chemical properties of substances involved in the immunoquantitation process, such as microspheres and antibodies, etc. In the present invention, we could see that shaking could significantly improve the sensitivity of T-Tau, p-Tau-181 and α-synuclein, but had no obvious effect on the sensitivity of Aβ1-40 and Aβ1-42. In order to improve the overall sensitivity, we needed to conduct shaking during the incubation process.

In order to further improve the sensitivity of Aβ1-40 and Aβ1-42, on the basis of the aforementioned incubation with shaking, we tried to add an additional incubating agent capable of reducing the settling of microspheres and promoting a full reaction into the incubation solution. The ingredients of the incubating agent were divided into polyol, a polymer and a nonionic surfactant according to the mode of action. For the polyol, sorbitol and glycerol were added; for the polymer, polyethylene and polyvinylpyrrolidone were added; for the nonionic surfactant, Tetronic1307, a Tween surfactant, a Triton surfactant and a polyoxyethylene stearate surfactant were added. Each reagent was formulated according to the molar concentration of the same kind of reagents in Example 1, and the others were carried out according to Example 1. The results were as shown in Table 13.

TABLE 13
Optimization of Sensitivity of addiction of Reagent (pg/mL)

| Reagent | | Aβ1-40 | Aβ1-42 | T-Tau | p-Tau-181 | α-synuclein |
|---|---|---|---|---|---|---|
| Blank | | 3.51 | 3.87 | 1.40 | 0.98 | 23.3 |
| Polyol | Sorbitol | 2.67 | 2.80 | 1.25 | 0.84 | 22.1 |
| | Glycerol | 3.14 | 3.18 | 1.33 | 0.91 | 22.7 |
| Polymer | Polyvinylidene | 2.45 | 2.68 | 1.31 | 0.88 | 22.7 |
| | Polyvinylpyrrolidone | 2.18 | 2.38 | 1.24 | 0.85 | 22.6 |
| Nonionic | Tetronic1307 | 2.28 | 2.48 | 1.18 | 0.81 | 22.7 |
| surfactant | Tween-80 | 2.19 | 2.59 | 1.24 | 0.88 | 22.6 |
| | Triton X-100 | 2.04 | 2.21 | 1.08 | 0.80 | 22.4 |
| | Polyoxyethylene stearate | 2.33 | 2.54 | 1.22 | 0.81 | 22.6 |

| | | | | | |
|---|---|---|---|---|---|
| Sorbitol + polyvinylpyrrolidone + | 1.50 | 1.69 | 0.88 | 0.53 | 21.2 |
| Triton X-100 | | | | | |

The results showed that: in the polyol, the sorbitol could effectively reduce the detection limits of Aβ1-40 and Aβ1-42; in the polymer, both polyethylene and polyvinylpyrrolidone could reduce the detection limits of Aβ1-40 and Aβ1-42, and polyvinylpyrrolidone had a better effect; each nonionic surfactant could reduce the detection limits of Aβ1-40 and Aβ1-42, among which Tetronic1307, Tween-80 and polyoxyethylene stearate had general effects, while Triton X-100 had a better effect. We added a combination of sorbitol, polyvinylpyrrolidone and Triton X-100 as an incubating agent, which played a role together to reduce the detection limits of the five detection indicators, but the detection limits of Aβ1-40 and Aβ1-42 were still high.

So we added a chelating agent on the basis of the combination of sorbitol, polyvinylpyrrolidone and Triton X-100. We screened a large number of chelating agents and had found that three chelating agents could improve the sensitivity of the five detection indicators of the present invention. The addition of the chelating agents and the determination of the detection limit described above were carried out according to the method of Example 1, and the results were as shown in Table 14.

TABLE 14
Optimization of Sensitivity of addiction of Reagent (pg/mL)

| Chelating agent | Aβ1-40 | Aβ1-42 | T-Tau | p-Tau-181 | α-synuclein |
|---|---|---|---|---|---|
| Blank | 1.50 | 1.69 | 0.88 | 0.53 | 21.2 |
| Sodium | 1.04 | 1.21 | 0.80 | 0.48 | 20.4 |
| citrate | | | | | |
| EDTA | 0.92 | 1.02 | 0.80 | 0.48 | 20.2 |
| Sodium | 0.98 | 1.14 | 0.83 | 0.51 | 20.8 |
| hyaluronate | | | | | |
| Sodium citrate + | 0.60 | 0.64 | 0.75 | 0.44 | 18.10 |
| sodium | | | | | |
| hyaluronate | | | | | |

The results showed that among sodium citrate, EDTA and sodium hyaluronate, sodium hyaluronate had the best effect in improving the sensitivity of each detection indicator, especially had a significant effect in reducing the detection limits of Aβ1-40 and Aβ1-42. Especially, in the present invention combinations of the above three ones were also tested, and it had been found that only sodium citrate and sodium hyaluronate could work together, so that the detection limits of the five detection indicators of the present invention were further reduced.

In principle, a polyol substance could increase the density of a preservation solution, so that the microspheres could exist in a suspension form, so as to reduce the probability of agglomeration and cross-linking of the microspheres, thereby in turn achieving the effect of promoting occurrence of the reaction, and avoiding the problem that because of agglomeration of immune microspheres or after a too long agglomeration time, the immune microspheres were firmly conjoined and were difficult to be dispersed again through shaking. The nonionic surfactant, also called an amphoteric surfactant, could be adsorbed on a site that was not completely closed on the surface of a coated immune microsphere, thereby improving the steric hindrance on the surface of the immune microsphere, further reducing the non-specific adsorption of other proteins, and meanwhile improving the dispersibility of the microsphere. It cooperated with the polyol to reduce the formation of microsphere agglomeration, thereby improving the accuracy and sensitivity of the detection result. The polymer could wrap or polymerize interfering proteins in serum and plasma samples together, thereby eliminating the interference with macromolecular proteins such as fibrin, making it impossible for impure proteins to interact with antigens/antibodies through weak interaction, and meanwhile reducing the possible non-specific antigen/antibody binding between samples and microspheres.

The chelating agent could promote the dispersion of microspheres and maintain a dispersed system structure, and cooperate with the aforementioned components to achieve a long-term effect of maintaining suspension of the microspheres, thereby improving the accuracy and sensitivity of the detection result, wherein sodium hyaluronate also had an additional effect: increasing the viscosity of an aqueous agent to increase the difficulty in deposition and attenuate the sedimentation rate, which made sodium citrate in cooperation with sodium hyaluronate produce the best effect.

### Example 4: Test for Screening Antibody Treatment

In immunoquantification, non-specific interference would have a great influence on detection. In the research process of the present invention, a non-specific sample was diluted and then tested, and the ratio of the tested concentration of the sample to the tested concentration of the stock solution was obviously different from the dilution multiple. The non-specific sample was tested by using an isotype control antibody, and the tested concentration of the non-specific sample was obviously higher than the background concentration, which indicated that the non-specific reaction was mainly induced by the Fc fragment of the antibody.

Therefore, in this example, a screening test of a method for treating an antibody was conducted, which tried to find out a method for treating an antibody, so that the detection could be avoided from the interference with a non-specific reaction to the greatest extent.

At first, we tried to add a passive blocking agent and an active blocking agent to block a nonspecific reaction, but the effect was general. Then we tried to remove the Fc fragment of the antibody, and the effect was the most obvious after the Fc fragment was removed. We carried out the aforementioned experiments respectively. The passive blocking agent was inactivated murine IgG, and the active blocking agent was an anti-HAMA polyclonal antibody, and the addition amount was the same as that in Example 1. The method of removing the Fc fragment was the same as that in Example 1. During the experiment of passive blocking and active blocking, only the corresponding passive or active blocking agent was added, and the Fc fragment was not removed from the antibody. During the experiment of removing the Fc fragment, the passive blocking agent and the active blocking agent were not added.

The non-specific sample used for testing was a disproportionate abnormal sample obtained after screening and dilution in the experiment. The sample was diluted with the sample diluting solution by a factor of 10, and loaded onto a machine to test the MFI value and the concentration value according to the method of Example 1, and the ratio of the concentration value of the stock solution to that of the diluted solution was calculated. The determination results of the concentration values of Aβ1-40, Aβ1-42, p-Tau-181 and T-Tau showed that the four detection indicators had the same trend. Here taking p-Tau-181 as an example, the results were as shown in Table 15.

TABLE 15
Concentration values of p-Tau-181 in nonspecific
sample under different antibody treatment methods

| | Concentration | Concentration | |
|---|---|---|---|
| | value of | value of | |
| Antibody treatment | stock solution | diluted solution | Proportion |
| Blank | 3.24 | 0.60 | 5.40 |
| Passive | 4.32 | 0.58 | 7.44 |
| blocking | | | |
| Active | 4.89 | 0.65 | 7.54 |
| blocking | | | |
| Removal of | 6.08 | 0.62 | 9.81 |
| Fc fragment | | | |

The experimental results showed that the determined concentrations of the stock solution and the diluted solution were close to the dilution ratio after the treatment of removing the Fc fragment, which indicated that the non-specific reaction hardly occurred and the detection sensitivity was high. The determination results of Aβ1-40, Aβ1-42 and T-Tau had the same trend as that of p-Tau-181, and the detection sensitivity was high.

However, the determination results of the MFI values of the stock solution and the diluted solution showed different trends when the α-synuclein antibody was subjected to the aforementioned different treatment, and the results were as shown in Table 16.

TABLE 16
MFI value of α-synuclein in non-specific sample under
different antibody treatment methods (tested for 10 times)

| | MFI of | MFI of | |
|---|---|---|---|
| Antibody treatment | Stock solution | diluted solution | Proportion |
| Blank | 4538.2 | 811.53 | 5.6 |
| Passive | 6741.57 | 801.37 | 8.4 |
| blocking | | | |
| Active | 7106.12 | 805.24 | 8.8 |
| blocking | | | |
| Removal of | 1320.89 | 158.86 | 8.3 |
| Fc fragment | | | |

According to the experimental results, the effect of removing the Fc fragment from α-synuclein had the similar interference removal effect to that of the addition of the blocking agent, but the MFI values of the stock solution and diluted solution were decreased and the sensitivity was decreased. Therefore, in the present invention, we did not remove the Fc fragment, but added a blocking agent into the reaction buffer to ensure the sensitivity of α-synuclein detection.

The possible reason was that for the α-synuclein antibody, the removal of the Fc fragment led to the change in the protein configuration of the α-synuclein antibody, which instead reduced the sensitivity of the α-synuclein antibody and led to the increase of error.

To sum up, in the present invention, Fc fragments were removed only from the Aβ1-40 antibody, the Aβ1-42 antibody, the p-Tau-181 antibody and the T-Tau antibody, and the Fc fragment of α-synuclein was not removed.

Although the present invention is disclosed above, the present invention is not limited thereto. Various changes and modifications can be made by those of skills in the art without departing from the spirit and scope of the present invention, and thus the claimed scope of the present invention should be based on the scope defined by the claims.

1. A kit for detecting an Alzheimer disease-related protein in human blood, comprising a solution of an antibody-conjugated microsphere, a biotin-conjugated an antibody and an incubating agent, wherein the incubating agent comprises a polyol, a polymer and a nonionic surfactant; the antibody is selected from one or more of the following antibodies: an antibody against Aβ1-40, an antibody against Aβ1-42, an antibody against p-Tau-181, an antibody against T-Tau, and an antibody against α-synuclein.

2. The kit according to claim 1, wherein the polyol is sorbitol, the polymer is polyvinylpyrrolidone, and the nonionic surfactant is Triton X-100.

3. The kit according to claim 1, wherein the antibody-conjugated microsphere is an A microsphere which is a 5 μm carboxyl microsphere, and the A microsphere comprises an A1 microsphere, an A2 microsphere, an A3 microsphere, an A4 microsphere and an A5 microsphere.

4. The kit according to claim 3, wherein the fluorescence intensity of each one of the A1-A5 microspheres was different.

5. The kit according to claim 3, wherein the antibody against Aβ1-40 is conjugated with the A1 microsphere, the antibody against Aβ1-42 is conjugated with the A2 microsphere, the antibody against p-Tau-181 is conjugated with the A3 microsphere, the antibody against T-Tau is conjugated with the A4 microsphere, and the antibody against α-synuclein is conjugated with the A5 microsphere.

6. The kit according to claim 3, wherein each antibody conjugated with the microsphere is a rabbit monoclonal antibody.

7. The kit according to claim 5, wherein the antibody against Aβ1-40, the antibody against Aβ1-42, the antibody against p-Tau-181 and the antibody against T-Tau do not contain a Fc fragment.

8. The kit according to claim 1, further comprising a fluorescent reagent which is biotin-BSA-SA-PE, wherein the biotin-BSA-SA-PE is a conjugate of streptavidin and phycoerythrin crosslinked with biotin-BSA.

9. The kit according to claim 1, further comprising a blocking agent, wherein the blocking agent is an inactivated murine IgG and an anti-HAMA polyclonal antibody.

10. The kit according to claim 1, further comprising a sample diluting solution, a reaction buffer, a washing buffer, a calibration product and a quality control, wherein the sample diluting solution comprises a tris(hydroxymethyl)aminomethane buffer, sodium chloride and Proclin300.

11. The kit according to claim 10, wherein the reaction buffer comprises the tris(hydroxymethyl)aminomethane buffer, sodium chloride, bovine serum albumin, a blocking agent, Proclin300 and Tween-20.

12. The kit according to claim 10, wherein the washing buffer comprises a phosphate buffer and Tween-20.

13. The kit according to claim 12, wherein the incubating agent comprises a tris(hydroxymethyl)aminomethane buffer, and the buffer comprises the sorbitol, polyvinylpyrrolidone and Triton X-100.

14. The kit according to claim 12, wherein the antibody is a combination of the antibody against Aβ1-40, the antibody against Aβ1-42 and the antibody against p-Tau-181.

15. The kit according to claim 12, wherein the antibody is a combination of the antibody against Aβ1-40, the antibody against Aβ1-42, the antibody against p-Tau-181 and the antibody against α-synuclein.

16. The kit according to claim 12, wherein the antibody is the antibody is a combination of the antibody against Aβ1-40, the antibody against Aβ1-42, the antibody against p-Tau-181, the antibody against T-Tau and the antibody against α-synuclein.

17. The kit according to claim 12, wherein the incubating agent further comprises a chelating agent, and the chelating agent is one of sodium citrate, EDTA and sodium hyaluronate.

18. The kit according to claim 17, wherein the chelating agent is sodium hyaluronate.

19. A method for detecting by using the kit according to claim 18, comprising the following steps:
S1: sequentially adding a reaction buffer, a solution of an antibody-conjugated microsphere and a sample to be tested into a sample tube;
S2: adding a biotin-conjugated antibody and an incubating agent into the mixture obtained in the step S1, and incubating the obtained mixture at 2-8° C. with protection from light and shaping;
S3: adding a fluorescent reagent into that mixture obtained in the step S2, and incubating the obtained mixture at room temperature with protection from light to obtain a first complex; and
S4: detecting the fluorescence type and fluorescence signal intensity of the first complex on a flow cytometer, and calculating to obtain the contents of Aβ1-42, Aβ1-40, T-Tau, p-Tau-181 and α-synuclein in the sample to be tested.

20. The method according to claim 19, wherein the sample to be tested is plasma.
